SEQUENCE LISTING (35 sequences)

SEQ ID NO: 1 - DNA, 59 bp, Artificial
  Bacterial
aaacgacggccagtgaattgtaatacgactcactataggcgcaaggaggtgatccagcc

SEQ ID NO: 2 - DNA, 16 bp, Escherichia coli
aaattgaagagtttga

SEQ ID NO: 3 - DNA, 66 bp, Escherichia coli
tcatggctcagattgaacgctggcggcaggcctaacacatgcaagtcgaacggtaacaggaagaag

SEQ ID NO: 4 - DNA, 86 bp, Escherichia coli
cttgcttctttgctgacgagtggcggacgggtgagtaatgtctgggaaactgcctgatggagggggataactactggaaacggtag

SEQ ID NO: 5 - DNA, 79 bp, Escherichia coli
ctaataccgcataacgtcgcaagaccaaagagggggaccttcgggcctcttgccatcggatgtgcccagatgggattag

SEQ ID NO: 6 - DNA, 35 bp, Escherichia coli
ctagtaggtggggtaacggctcacctaggcgacga

SEQ ID NO: 7 - DNA, 7 bp, Escherichia coli
tccctag

SEQ ID NO: 8 - DNA, 361 bp, Escherichia coli
ctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaag

SEQ ID NO: 9 - DNA, 56 bp, Escherichia coli
cttgagtctcgtagaggggggtagaattccaggtgtagcggtgaaatgcgtagaga

SEQ ID NO: 10 - DNA, 155 bp, Escherichia coli
tctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggag

SEQ ID NO: 11 - DNA, 206 bp, Escherichia coli
ctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagaatgagaatgtgccttcgggaaccgtgagacaggtgctgcatggctgtcgtcag

SEQ ID NO: 12 - DNA, 289 bp, Escherichia coli
ctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtgga

SEQ ID NO: 13 - DNA, 85 bp, Escherichia coli
tcagaatgccacggtgaatacgttcccgggccttgtacacaccgcccgtcacaccatgggagtgggttgcaaaagaagtaggtag

SEQ ID NO: 14 - DNA, 89 bp, Escherichia coli
cttaaccttcgggagggcgcttaccactttgtgattcatgactggggtgaagtcgtaacaaggtaaccgtaggggaacctgcggttgga

SEQ ID NO: 15 - DNA, 11 bp, Escherichia coli
tcacctcctta

SEQ ID NO: 16 - DNA, 8 bp, Vibrio proteolyticus
gagtttga

SEQ ID NO: 17 - DNA, 179 bp, Vibrio proteolyticus
tcatggctcagattgaacgctggcggcaggcctaacacatgcaagtcgagcggaaacgagttatctgaaccttcggggaacgatatcggcgtcgagcggcggacgggtgagtaatgcctgggaaattgccctgatgtgggggataaccattggaaacgatggctaataccgcataatag

SEQ ID NO: 18 - DNA, 62 bp, Vibrio proteolyticus
cttcggctcaaagagggggaccttcgggcctctcgcgtcaggatatgcccaggtgggattag

SEQ ID NO: 19 - DNA, 35 bp, Vibrio proteolyticus
ctagttggtgaggtaagggctcaccaaggcgacga

SEQ ID NO: 20 - DNA, 7 bp, Vibrio proteolyticus
tccctag

SEQ ID NO: 21 - DNA, 17 bp, Vibrio proteolyticus
ctggtctgagaggatga

SEQ ID NO: 22 - DNA, 400 bp, Vibrio proteolyticus
tcagccacactggaactgagacacggtccagactcctacgggaggcagcagtggggaatattgcacaatgggcgcaagcctgatgcagccatgccgcgtgtgtgaagaaggccttcgggttgtaaagcactttcagtcgtgaggaaggtagtgtagttaatagatgcattatttgacgttagcgacagaagaagcaccggctaactccgtgccagcagccgcggtaatacggagggtgcgagcgttaatcggaattactgggcgtaaagcgcatgcaggtggtgtgttaagtcagatgtgaaagcccggggctcaacctcggaatagcatttgaaactggcagactagagtactgtagaggggggtagaatttcaggtgtagcggtgaaatgcgtagaga

SEQ ID NO: 23 - DNA, 155 bp, Vibrio proteolyticus
tctgaaggaataccggtggcgaaggcggccccctggacagatactgacactcagatgcgaaagcgtggggagcaaacaggattagataccctggtagtccacgccgtaaacgatgtctacttggaggttgtggccttgagccgtggctttcggag

SEQ ID NO: 24 - DNA, 207 bp, Vibrio proteolyticus
ctaacgcgttaagtagaccgcctggggagtacggtcgcaagattaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcag

SEQ ID NO: 25 - DNA, 290 bp, Vibrio proteolyticus
ctcgtgttgtgaaatgttgggttaagtcccgcaacgagcgcaacccttatccttgtttgccagcacgtaatggtgggaactccagggagactgccggtgataaaccggaggaaggtggggacgacgtcaagtcatcatggcccttacgagtagggctacacacgtgctacaatggcgcatacagagggcggccaacttgcgaaagtgagcgaatcccaaaaagtgcgtcgtagtccggattggagtctgcaactcgactccatgaagtcggaatcgctagtaatcgtgga

SEQ ID NO: 26 - DNA, 105 bp, Vibrio proteolyticus
tcagaatgccacggtgaatacgttcccgggccttgtacacaccgcccgtcacaccatgggagtgggctgcaaaagaagtgggtagtttaaccttcgggaggacgc

SEQ ID NO: 27 - RNA, 19 nt, Artificial
  Example RNA oligo or fragment thereof for mass spectrometric identification
ccccuugauagccgcuacg

SEQ ID NO: 28 - RNA, 3 nt, Artificial Sequence;
  Example RNA oligo or fragment thereof for mass spectrometric identification
ccg

SEQ ID NO: 29 - RNA, 4 nt, Artificial Sequence;
  Example RNA oligo or fragment thereof for mass spectrometric identification
cccc

SEQ ID NO: 30 - RNA, 4 nt, Artificial Sequence;
  Example RNA oligo or fragment thereof for mass spectrometric identification
auag

SEQ ID NO: 31 - RNA, 5 nt, Artificial Sequence;
  Example RNA oligo or fragment thereof for mass spectrometric identification
cuacg

SEQ ID NO: 32 - RNA, 6 nt, Artificial Sequence;
  Example RNA oligo or fragment thereof for mass spectrometric identification
cccuug

SEQ ID NO: 33 - RNA, 8 nt, Artificial Sequence;
  Example RNA oligo or fragment thereof for mass spectrometric identification
ccgcuacg

SEQ ID NO: 34 - RNA, 11 nt, Artificial Sequence;
  Example RNA oligo or fragment thereof for mass spectrometric identification
ccccuugauag

SEQ ID NO: 35 - RNA, 12 nt, Artificial Sequence;
  Example RNA oligo or fragment thereof for mass spectrometric identification
auagccgcuacg

(19) United States
(12) Patent Application Publication
(10) Pub. No.: US 2005/0142584 A1
(43) Pub. Date: Jun. 30, 2005

(54) Microbial identification based on the overall composition of characteristic oligonucleotides

(76) Inventors: Richard C. Willson, Houston, TX (US); George E. Fox, Houston, TX (US); Zhang Zhengdong, Houston, TX (US); George W. Jackson, Houston, TX (US)

Correspondence Address: JAMES D. PETRUZZI, 4900 WOODWAY SUITE 745, HOUSTON, TX, 77056, US

(21) Appl. No.: 10/955,990

(22) Filed: Sep. 30, 2004

Related U.S. Application Data
(60) Provisional application No. 60/507,589, filed on Oct. 1, 2003.

(51) Int. Cl. C12Q 1/68

(52) U.S. Cl. 435/6

(57) ABSTRACT

Identification of microorganisms based on the sequences of their 5S, 23S and particularly 16S ribosomal RNAs is growing in utility as the database of known ribosomal RNA sequences expands. Experimental identification is usually based on matching the experimentally-determined sequence of an organisms rRNA to a previously-determined sequence in the databank, or hybridization of the organisms rRNA or encoding rDNA to an oligonucleotide probe specific for an organism anticipated to be present in the sample. Here we propose the identification of microorganisms based on the overall composition (not sequence or hybridization propensity) of characteristic molecules derived from their rRNA or rDNA sequences by enzymatic cleavage or localized amplification. Ribonuclease T1 fragments of rRNA composition determination by mass spectrometry are especially favored. The characteristic molecules used can be chosen to be “compositional signatures” whose presence/absence is known to be associated with particular groups of organisms.

### CROSS REFERENCE TO RELATED APPLICATIONS

The present application is related to the following U.S. patent application: provisional patent application No. 60/507,589 titled “Microbial Identification Based on the Overall Composition of Characteristic Oligonucleotides” filed Oct. 1, 2003, which is hereby incorporated by reference as if fully set forth herein.

### STATEMENT REGARDING FEDERALLY SPONSORED RESEARCH OR DEVELOPMENT

### DESCRIPTION OF ATTACHED APPENDIX

Not Applicable

### BACKGROUND OF THE INVENTION

1. Field of the Invention

The present invention relates to the general fields of biotechnology, microbiology and clinical diagnosis and more particularly to methods and systems for identifying microorganisms without sequencing or the use of probes.

2. Description of the Background Art

Conventional determinative bacteriology traditionally relied on the characterization of phenotypic traits of pure cultures obtained from specimens after cultivation and isolation of bacteria on appropriate laboratory media [Wintzingerode, Fvon, et al. PNAS May 14, 2002 vol. 99 no. 10 7039-7044]. The ever-increasing amount of sequence data from bacterial organisms has made various molecular approaches more tenable. Common examples of such approaches include comparative sequencing of PCR-amplified 16S ribosomal RNA genes (rDNA), isotopic or fluorescently labeled hybridization probes (molecular beacons), or reverse transcription of ribosomal RNA (rRNA) and amplification (RT-PCR, or “Eberwine-type” amplification) used in conjunction with hybridization probes or sequencing. Currently, 16S rRNA or the genes thereof (rDNA) comprise the largest set of gene-specific sequence data. However, relevant information for other targets including 5S rRNA, 23S rRNA, rRNA spacer regions and RNase P RNA is also accumulating rapidly, in part because of complete genome sequencing efforts.

Drawbacks exist to sequencing and hybridization-based methods, however. Sequencing by capillary electrophoresis can be time consuming and is generally not amenable to mixtures of oligonucleotides from multiple organisms. Capillary electrophoresis devices can also be delicate and not appropriate for field use, e.g. remote sites of biological interest and extraterrestrial locations. Detection of a microorganism by a hybridization probe implies a priori knowledge of a putative characteristic sequence and therefore may be limited in generality when assaying an unknown sample. Microarrays for phylogenetic typing have certainly been described, but sample labeling and hybridization may require 18 hours or more in many cases. FRET-based probes deployed in free-solution often referred to as “hairpin probes” or molecular beacons also, and obviously, require a priori design of a putative complimentary sequence being assayed.

### BRIEF SUMMARY OF THE INVENTION

An advantage of the invention is to create speed and accuracy of organism identification or classification without the use of complete sequencing of a molecule or fragments thereof.

Another advantage of the invention is to provide identification without the inclusion of highly organism-specific hybridization probes in the assay.

Another advantage of the invention is to provide a means for disregarding a high background of contaminating or uninteresting compositions, thereby facilitating identification or classification of a minority organism.

Another advantage of the invention is to provide a system that continually analyzes and increases the knowledge base of the frequency and distribution of characteristic oligonucleotide fragments or proteins among living organisms.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a method for systematically sampling a bacterial or viral population.

In accordance with a preferred embodiment of the invention, there is disclosed a system for isolating or selectively amplifying a nucleic acid molecule.

In accordance with a preferred embodiment of the invention, there is disclosed a process for performing mass-spectrometric analysis of the characteristic compositions rendered from some enzymatic or chemical fragmentation or selective amplification of the nucleic acid.

In accordance with a preferred embodiment of the invention, there is disclosed a method for comparing the resulting fragment compositions with those of signature sequences predicted from sequence database information.

In accordance with a preferred embodiment of the invention, there is disclosed a method for using statistical methods to give a confidence index that a given organism or multiple organisms is/are present in the sample.

In accordance with a preferred embodiment of the invention, there is disclosed a method for identifying or detecting organisms such as bacteria, eukaryotes, archaebacteria, or viruses having the steps of isolating a characteristic nucleic acid or protein component of an organism, determining at least a portion of the monomer composition of a sequence derived from the characteristic nucleic acid or protein; and identifying or detecting the micro-organism from which the characteristic nucleic acid or protein was derived by reference to a database of compositions of nucleic acids and proteins produced by organisms.

In accordance with a preferred embodiment of the invention, there is disclosed a system for identifying or detecting organisms such as bacteria, viruses, archaebacteria or eukaryotes having a chemical isolator or amplifier for identifying the characteristic nucleic acid or protein of an organism present in a specimen, a controlled fragmentation reactor that generates sub-fragments of the characteristic acid or protein, a mass spectrometer that measures the molecular weight of the sub-fragments and generates a set of representative data, a computer that processes said data and compares the measured weights with known predicted sub-fragment masses to make an identification.

In accordance with a preferred embodiment of the invention, there is disclosed a method for identifying or detecting organisms such as bacteria, eukaryotes, archaebacteria, or viruses having the steps of determining known fragment sequences for a pre-determined set of nucleic acid or proteins, isolating a characteristic nucleic acid or protein component of an organism present in a specimen, determining at least a portion of the monomer composition of a sequence derived from the characteristic nucleic acid or protein; and identifying or detecting the micro-organism from which the characteristic nucleic acid or protein was derived by reference to a database of compositions of nucleic acids and proteins produced by organisms.

### BRIEF DESCRIPTION OF THE DRAWINGS

FIG. 1 shows a Matrix Assisted Laser Desorption Ionization Time of Flight, or MALDI-TOF spectrum of a T1 ribonuclease digest of synthetic 19mer RNA oligonucleotide in accordance with a preferred embodiment of the invention.

FIG. 2 shows a calculated distribution of oligonucleotides according to the their lengths from a population of 1,921 organisms generated by RNase T1 and RNase A digestion of 16S rRNA in accordance with a preferred embodiment of the invention.

FIG. 3 shows an idealized mass spectrum from an in silico digest of E. coli 5S ribosomal RNA in accordance with a preferred embodiment of the invention.

FIG. 4 assists in the discussion of one possible computational scheme for comparing an experimentally observed mass spectrum to lists of organisms who may have contributed the observed mass or peak.

The drawings constitute a part of this specification and include exemplary embodiments to the invention, which may be embodied in various forms. It is to be understood that in some instances various aspects of the invention may be shown exaggerated or enlarged to facilitate an understanding of the invention.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The present invention encompasses, among other things, any system which:
1) systematically samples a bacterial or viral population
2) isolates or selectively amplifies a nucleic acid molecule
3) performs mass-spectrometric analysis of the characteristic compositions rendered from some enzymatic or chemical fragmentation or selective amplification of the nucleic acid.
4) Compares the resulting fragment compositions with those of signature sequences predicted from sequence database information
5) Uses statistical methods to give a confidence index that a given organism or multiple organisms is/are present in the sample

Although small subunit ribosomal RNA (16S) sequences have historically been used most often for phylogenetic typing and evolutionary relatedness, it is beneficial to extend these ideas to other informative molecules and sequence spaces in the genome or it's transcripts that may have “characteristic” or “signature” utility for a given organism. The terminology “signature sequence” is used herein to specify oligonucleotides or oligodeoxynucleotide sequences carrying useful information regarding genetic affinity of the organism in which the sequence fragment resides [McGill T J, Jurka J, Sobieski J M, Pickett M H, Woese C R, Fox G E. “Characteristic archaebacterial 16S rRNA oligonucleotides.” Syst Appl Microbiol. 1986; 7: 194-197., 1986; Zhang et al., 2002]. In other words, a single characteristic oligonucleotide need not be a uniquely present in the organism or group of organisms for which it is an indicator. It should be noted that such signature sequences are distinct from the probes or “signature” probes that are commonly employed in hybridization, PCR, or microarray assays. The latter are typically required to be uniquely present in the target organism or organism group that they specify. In this description of the invention, we will use the term “Information Containing Molecule” or ICM for any starting material such as 16S ribosomal RNA that is under selective or functional pressure leading to non-random distribution of nucleotides at certain positions in a sequence.

The present invention discloses that there are actually signature or characteristic compositions that can provide unique identifying information for organisms. By adding up the molecular masses of the monomers comprising signature sequences, it is shown herein that there is identifying information in signature compositions (masses) which are readily calculable prior to performing any assay for their presence. The measurement of composition alone results in degeneracy and loss of information, e.g. a nucleic acid fragment AAACG is indistinguishable by mass from AACAG. Regardless, we have demonstrated that unique mass identifiers, either taken alone, or by detecting the presence of multiple fragments of certain molecular mass, can uniquely identify an organism, or in the very least phylogenetically type that organism to a highly useful degree.

The present invention provides for the rapid identification of bacteria, without using probes or sequencing. This invention proposes the use of mass spectrometry to rapidly identify the presence of signature or “characteristic” oligonucleotides in isolates from pure culture or a complex mixture of organisms. It has previously been demonstrated that large numbers of highly informative signature sequences exist in the 16S rRNA database and algorithms have been developed for identifying them [Zhang, Z, Willson, R C, Fox, G E, “Identification of Characteristic Oligonucleotides in the 16S Ribosomal RNA Sequence Dataset”, Bioinformatics, 2002; 18: 244-250]. Furthermore, it is disclosed that there are not only signature or characteristic sequences, but rather compositions. These compositions, taken either independently, or when multiple masses are taken in conjunction, have identifying power. Monomers typically are not randomly distributed in the characteristic ICM. The fact that there is selective pressure for an organism to have a functional ribosome, for example, results in characteristic sub-fragments of the molecule. Any other molecule having the same quality could be used to generate catalogues of characteristic sequences and compositions. Examples would be the other two ribosomal RNA fragments, 5 and 23S, RNase P, etc. Although databases of such sequences could be developed privately, public databases of such sequences exist. Examples are the Ribosomal Database Project (both 1 and 2) [Maidak, et al. “The Ribosomal Database Project Continues” Nucleic Acids Research, 2000, vol. 28, no. 1,173-174], NCBI databases, GenBank, and any public genome sequencing project. Some example web addresses for such projects are, in no particular order:
http://rdp.cme.msu.edu/
http://135.8.164.52/html/
http://prion.bchs.uh.edu/Signature16S/index.html
http://ncbi.nlm.nih.gov
http://prion.bchs.uh.edu/16S_signatures/

In a preferred embodiment, in silico, or computer-simulated, digestions of the target RNA by endoribonucleases are performed to predict resultant compositions (RNA fragment masses). In other embodiments, however, the RNA may be fragmented by any other reproducible, predictable manner so long as the in vitro or in vivo fragmentation experiment can be simulated by the computer and the resultant masses catalogued. Even the ionization event in the mass spectrometer itself and/or interaction with the MALDI matrix could be used to predictably and reproducibly generate signature compositions. One or multiple restriction enzymes may be used to digest rDNA (cDNA to rRNA) or genomic DNA. The resulting characteristic compositions can be used to “mass fingerprint” the presence of single or multiple organisms, by comparing the predicted compositions with MALDI-TOF mass spectra of the digests, the mass spectrum can be used to assign genetic affinity to an organism, thereby placing the organism on the “tree of life” or at least showing some evolutionary relation to other organisms. Applications include detection and identification of pathogenic organisms in clinical samples and food, as well as for use in biodefense. The method may also find application in virus and cell typing, as it will become increasingly useful as additional advances in database size and mass spectrometry technology occur. It should also be emphasized that the invention is not limited to the detection of presence or absence of an organism, but comprises the concepts of genetic affinity to taxonomically/phylogenetically type an organism even if that exact organism is previously unknown. In this manner, the invention is a departure from simple empirical matching of a DNA restriction fingerprint to another as in Restriction Fragment Length Polymorphism (RFLP) or similar methods such as AFLP. The invention described herein will be able to put the organism's identification into taxonomical context. Methods for generating most-parsimonious trees or phylogenetic dendrigrams are well known. Once the organism identity or some quotient of relatedness to previously known organisms is established, the organism observed can be placed on a phylogenetic tree.

There are several likely implementations of the invention. Although many bacteria are unculturable, ribosomal RNA has the advantage of being naturally present in multiple copies. This means that, depending on the detection limits of the mass spectrometer, it may be possible to isolate enough of the characteristic molecule (16S rRNA in one embodiment) to perform a digest and mass-fingerprint the organism without any type of nucleic acid amplification. For example, isolation of total RNA from a small culture using standard methods would be carried out [Chomczynski P, Sacchi N: Single-step method of RNA isolation by acid guanidinium thiocyanate-phenol-chloroform extraction. Anal Biochem 1987, 162: 156-159] and [Sambrook J, Fitsch E F, Maniatis T: Molecular Cloning: A Laboratory Manual. Cold Spring Harbor, Cold Spring Harbor Press 1989].

Chomczynski has also described isolation of DNA, RNA, and Protein fractions, each of which may be used in this invention, either alone or in conjunction, as information-containing biological fractions.

Typically, 90-97% of the total nucleic acid content following this isolation comprises the following: the transfer RNAs, or “4S”, and 5S, 16S, and 23S rRNA. From this mixture is isolated the ICM of choice, e.g. 16S rRNA. This could be performed by any acceptable chromatographic, affinity such as lysine sepharose, immobilized bead, electrophoresis, capillary electrophoresis, electrophoresis combined with gel extraction or other method known to those skilled in the art. Complete RNase T1 digestion of E. coli 16S rRNA results in 488 fragments with no internal G residues, many of which are degenerate in mass but some of which may be uniquely identifying depending on sample source or context. Below is a simple example MATLAB code for calculating fragment masses from a complete ribonuclease T1 digestion of an input sequence.

Example MATLAB Code for Generating Ribonuclease T1 Fragments from a Single Input Sequence.

```
function [threeprimePO4unique] = T1digestion_avgmasses(sequence,pattern)
%======================================================================
% Mass Spec Tools for MATLAB
%
% “In Silico” Ribonuclease T1 digestion of imported sequence
% Use “File -> Import Data at MATLAB command window to import .xls file
% Sequence must be in single column in .xls file
%
%
%======================================================================
% [f] = xlsread(‘whateverinputsequence.xls’)
format long g;
A=65; % ASCII Text values in double precision
C=67;
G=71;
T=84;
U=85;
‘Length of Sequence’
n=length(sequence)
for m=1:n % n is length of oligo
newseq(m,1)=sequence{m,1}; % conversion from cellarray to chararray
end
newseq=double(newseq); % conversion to double prec values
% average masses
for m=1:n
  if newseq(m,1)==A
    newseq(m,1)=329.2091;
  elseif newseq(m,1)==C
    newseq(m,1)=305.1840;
  elseif newseq(m,1)==G
    newseq(m,1)=345.2084; % ***** cutting site *****
  elseif newseq(m,1)==T
    newseq(m,1)=320.1843;
  elseif newseq(m,1)==U
    newseq(m,1)=306.1687;
  end
end
‘The mass of the entire sequence (3prime-PO4) is:’
masssum_seq=sum(newseq)+17.0027
newseq % sequence in mass form
% pattern = input(‘Enter Methylation pattern vector? - for no methylation enter “zeros(n,1)” ’);
methyl=14.0156
newseq=newseq+pattern*methyl
%%%%%%%%%%%%%%%%%%%%%%%%%%%%%%%%%%%%%%%%%%%%%%%%%%%%%%%%%
%%%%%%%%%%%
% T1 digestion algorithm (masses):
i=1;
A=zeros(n+1,i);
for m=1:n % “frag's” are from start up to nth G
  if (n==m)&(newseq(m,1)==345.2084)
    i=i;
  elseif newseq(m,1)==345.2084
    frag=newseq(1:m,1);
    frag(n+1,1)=zeros;
    A(:,i)=frag;
    i=i+1;
  else
    frag=newseq(1:n,1);
    frag(n+1,1)=zeros;
    A(:,i)=frag;
  end
end
A; % represents 5′ fragments with pieces lost from 3′ end (some of the possible incomplete digestion products)
x=1:i; % row vector
x=x′; % col “”
longfiveprimefragsPO4=[x sum(A(:,x))′];
longfiveprimefragsPO4(:,2)=longfiveprimefragsPO4(:,2)+17.0027; % ADDING OH to 5′ end, results in net
negative −1 for MALDI
% longfiveprimefragsOH=[x longfiveprimefragsPO4(:,2)−79.9662]; % Subtracting HPO3
longfiveprimefragscyclicPO4=[x longfiveprimefragsPO4(:,2)−18.0105];
%
% Now calculate all small pieces
for q=2:i
  for z=1:q−1
    A(:,q)=A(:,q)−A(:,z);
  end
end
%%%%%%%%%%%%%%%%%%%%%%%%%%%%%%%%%%%%%%%%%%%%%%%%%%%%%%%%%
%%%%%%%%%%%%%% END DIGEST
‘The number of digestion fragments is’, i
A
%frag masses 5′ to 3′?? check on order
x=1:i; % row vector
x=x′; % col “”
fragmasses=[x sum(A(:,x))′];
threeprimePO4=[x fragmasses(:,2)+17.0027]; % ADDING OH to 5′ end, results in net negative −1 for MALDI
% threeprimeOH=[x threeprimePO4(:,2)−79.9662]; % SUBTRACTING HPO3
threeprimecyclic=[x threeprimePO4(:,2)−18.0105];
peaks=ones(i,1);
PO4=sort(threeprimePO4);
% Parse duplicate masses in PO4 peaks
p=1;
for n=1:i−1
  if PO4(n,2)˜=PO4(n+1,2)
    threeprimePO4unique(p,1)=PO4(n,2);
    p=p+1;
  end
end
threeprimePO4unique(p,1)=PO4(i,2); % get last mass
threeprimePO4unique; % unique PO4 terminated peaks
threeprimePO4plusSodium=threeprimePO4unique+21.9819; % ADDING Na, losing an H to compensate
% cyclic=sort(threeprimecyclic);
% % Parse duplicate masses in 2′-3′ cyclic PO4 peaks
% p=1;
% for n=1:i−1
% if cyclic(n,2)˜=cyclic(n+1,2)
% threeprimecyclicunique(p,1)=OH(n,2);
% p=p+1;
% end
% end
% threeprimecyclicunique(p,1)=OH(i,2); % get last mass
% threeprimecyclicunique; % unique 2′-3′ cyclic PO4 terminated peaks
% threeprimecyclicplusSodium=threeprimecyclicunique+21.9819; % ADDING Na, losing an H to compensate
for neg. MALDI charge
cyclic=sort(threeprimecyclic);
% Parse duplicate masses in 2′-3′ cyclic PO4 peaks
p=1;
for n=1:i−1
  if cyclic(n,2)˜=cyclic(n+1,2)
    cyclicPO4unique(p,1)=cyclic(n,2);
    p=p+1;
  end
end
cyclicPO4unique(p,1)=cyclic(i,2); % get last mass
cyclicPO4unique; % unique OH terminated peaks
cyclicPO4plusSodium=cyclicPO4unique+21.9819; % ADDING Na, losing an H to compensate for neg. MALDI
charge
header=‘cyclicPO4unique cyclicPO4plusSodium threeprimePO4unique threeprimePO4plusSodium’
Summary=[cyclicPO4unique cyclicPO4plusSodium threeprimePO4unique threeprimePO4plusSodium]
figure;
bar(threeprimecyclic(:,2),peaks,0.0)
  xlabel(‘m/z’);
  ylabel(‘peak height=“1”’);
  title(‘“mass spec” for 5prime-OH, 2prime-3prime cyclic phosphate’);
figure;
bar(threeprimePO4(:,2),peaks,0.0)
  xlabel(‘m/z’);
  ylabel(‘peak height=“1”’);
  title(‘“mass spec” for 5primeOH,3prime terminal-PO4’);
figure;
hist(threeprimecyclic(:,2),length(threeprimecyclic))
  title(‘Histogram for 5primeOH,3prime OH fragments’);
figure;
hist(threeprimePO4(:,2),length(threeprimePO4))
        title(‘Histogram for 5primeOH,3prime PO4 fragments’);
```

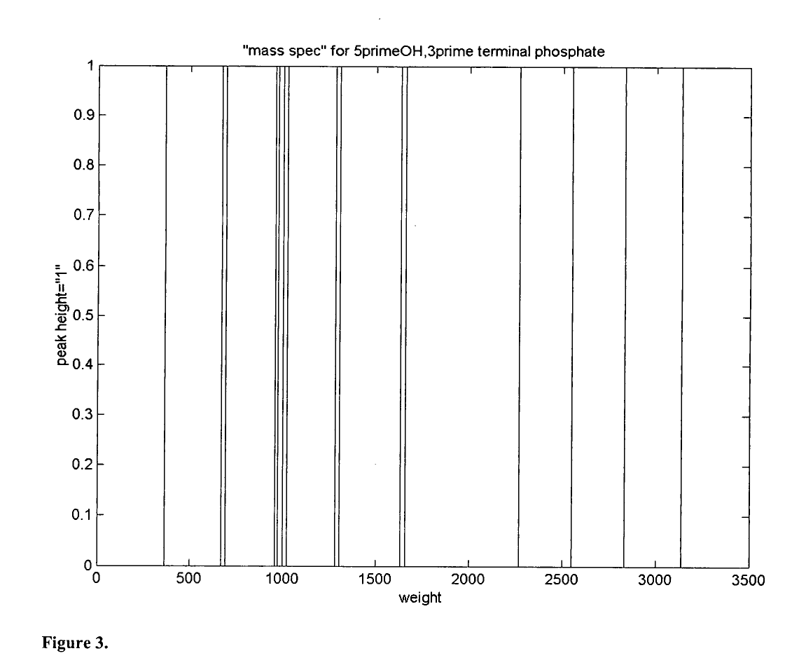

The above program arbitrarily assigns a peak height of “1” to every fragment in the spectrum. An example of the output of this program is shown in FIG. 3. The program input was the 120 base sequence for 5S rRNA from E. coli. In list format the output is of this form:

ans =
The number of digestion fragments is
i =
42
threeprimePO4 =

| | |
|---|---|
| 1 | 669.3811 |
| 2 | 1279.7491 |
| 3 | 363.2124 |
| 4 | 668.3964 |
| 5 | 363.2124 |
| 6 | 997.6055 |
| 7 | 998.5902 |
| 8 | 668.3964 |
| 9 | 668.3964 |
| 10 | 363.2124 |
| 11 | 669.3811 |
| 12 | 363.2124 |
| 13 | 2830.6789 |
| 14 | 2548.5353 |
| 15 | 973.5804 |
| 16 | 2267.3764 |
| 17 | 1021.6306 |
| 18 | 669.3811 |
| 19 | 1656.0237 |
| 20 | 973.5804 |
| 21 | 998.5902 |
| 22 | 668.3964 |
| 23 | 973.5804 |
| 24 | 998.5902 |
| 25 | 363.2124 |
| 26 | 998.5902 |
| 27 | 669.3811 |
| 28 | 669.3811 |
| 29 | 363.2124 |
| 30 | 363.2124 |
| 31 | 363.2124 |
| 32 | 3136.8476 |
| 33 | 668.3964 |
| 34 | 692.4215 |
| 35 | 692.4215 |
| 36 | 998.5902 |
| 37 | 363.2124 |
| 38 | 363.2124 |
| 39 | 1632.9833 |
| 40 | 1302.7895 |
| 41 | 363.2124 |
| 42 | 958.5658 |

Many of these 42 T1 fragments are degenerate. Sorted, the unique masses are:
threeprimePO4unique
363.2124
668.3964
669.3811
692.4215
958.5658
973.5804
997.6055
998.5902
1021.6306
1279.7491
1302.7895
1632.9833
1656.0237
2267.3764
2548.5353
2830.6789
3136.8476

The actual numbers are dependent on the MALDI mode assumed when the program is executed, e.g. negative or positive ion mode, and somewhat arbitrary up to the limits of resolution between distinct compositions and may contain significant digits beyond the limit of current spectrometers. While this example only has utility of calculating fragment masses for one sequence, similar subroutines have been employed by the inventors to calculate the RNase T1 fragment masses for many hundreds of sequences from the Ribosomal Database Project. Average molecular masses were used in the above example, but it may be beneficial to use the monoisotopic masses in the calculation. Commercial MALDI-TOF software packages often have the ability to fold isotopic distributions into their parent, monoisotopic mass, simplifying the spectra when it is possible to obtain the requisite resolution.

Once characteristic fragment mass calculations are made on one, many, or all available sequences (often filtered to meet certain completeness criteria), these calculated mass-fingerprints or bar-codes can be used to compare to experimental mass spectra. The invention described herein may rely on methods for simplifying spectra based on de-noising, smoothing or averaging, isotopic distribution analysis, baseline correction, or any other common methods available to mass spectrometrists skilled in the art. Once the experimental mass spectrum peaks exist, that is, they meet the above criteria and have sufficient signal-to-noise to be considered “real” peaks present in the sample, experimental spectra are compared to the predicted.

Computations regarding the use of multiple peaks are dependent on the number of sequences taken into consideration for purposes of fragment generation. In one embodiment a simple quotient system can be employed to generate an index or probability as to whether a certain organism was present in the sample. The following is an explanation of a data analysis simulation carried out by the inventors. “Each molecular weight in this collection may be attributed to a number of organisms whose 16S rRNAs digested by the RNase can generate one or several different oligonucleotides of the same molecular weight. The entire set of organisms identified by all the molecular weights and the number of times with which each of the organisms is identified are recorded. The probability that an organism is present in the sample is calculated as the ratio of the frequency with which it is identified to the number of oligonucleotides of different molecular weights in its RNase T1 catalogue of 16S rRNA. In the end, the program gives the list of all the organisms that are probably present in the sample and the corresponding probabilities.”

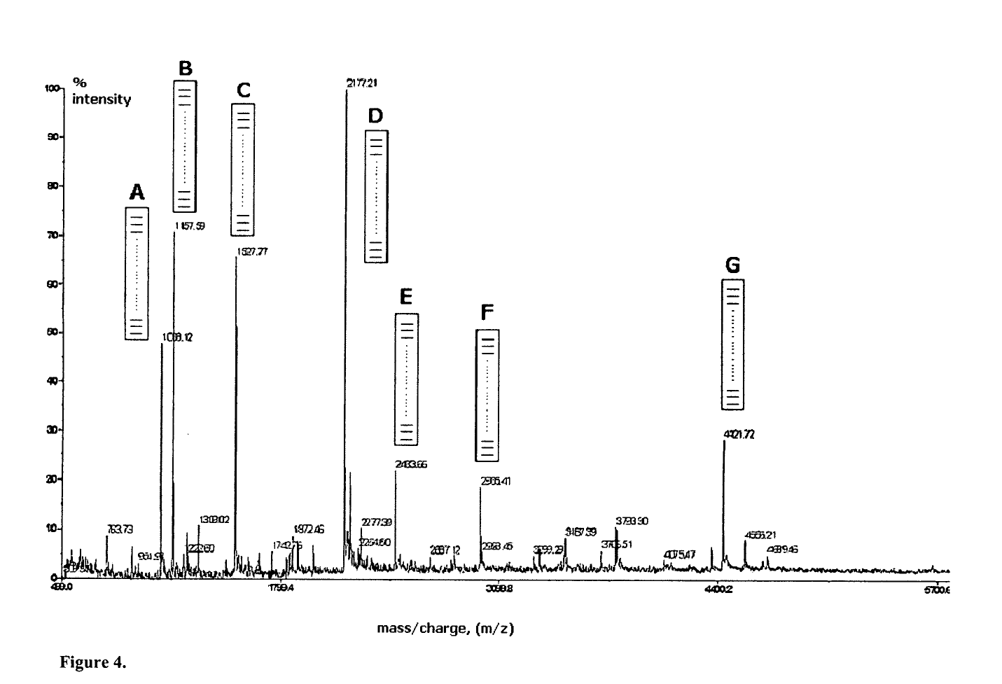

Another approach is illustrated in FIG. 4. This approach assumes that no peaks or compositions are falsely present in the observed spectrum. FIG. 4 shows a simplified situation for illustrative purposes. For each peak (mass m₁ to m₇) observed in the spectra, a list is generated from previous calculations of all possible “owners” or contributors of that peak. In FIG. 4 a list of organisms, A through G is generated for each of seven peaks. In practice, every peak present in the observed spectrum or spectra meeting signal to noise requirements would generate an organism list, but for clarity we have shown only lists A through G. Let lists A through G identify the following possible mass contributors:

| A | B | C | D | E | F | G |
|---|---|---|---|---|---|---|
| Bob | Bob | Charley | Bob | All known | Elvis | Bob |
| Harry | Elvis | David | Charley | organisms | | Charley |
| Sue | Frank | Frank | | contribute | | Harry |
| Tim | | Tim | | this mass | | Sue |
| Zora | | | | | | |

Note that Tim and Zora are underlined. Referring to FIG. 4, an absence of a peak at 5000 Daltons which Tim and Zora are calculated to contribute means that they are removed from any other lists on which they might be known owners. It is important to note that each list will likely have a different number of organisms, n₁ to n₇. These numbers are likely to vary widely in magnitude. If m₆ is a uniquely identifying mass, present in only one organism for example, then n₆=1, and list F will be a short one containing only one organism name. The other six lists, however might vary in length from 2 to N, where N is the number of all sequenced organisms used to generate the mass fragment catalogues). It is also worth note that although Elvis has a unique identifier represented by peak, m₆, he appears in lists B and E. The intersection, of the lists, may be used to generate sublists. Taking just pair wise intersections.
A B=[Bob]
A C=[nullset or Tim]
A D=[Bob]
A E=[Bob, Harry, Sue, Tim, Zora]
A F=[nullset]
A G=[Bob, Harry, Sue]
B C=[Frank]
B D=[Bob]
B E=[Bob, Elvis, Frank]
B F=[Elvis]
B G=[Bob]
C D=[Charley]
C E=[Charley, David, Frank, Tim]
C F=[nullset]
C G=[Charley]
D E=[Bob, Charley]
D F=[nullset]
D G=[Bob, Charley]
E F=[Elvis]
E G=[Bob, Charley, Harry, Sue]
F G=[nullset]

Any intersection of list N with E is the same as N. But in this rudimentary example it can be seen that the list lengths are quickly reduced.

A E B or any other 3 way intersection with E yields the same result as ignoring E.

Taking all 2 way intersections which did not reduce to a single member and intersecting them with the other lists,
A G=[Bob, Harry, Sue] B=[Bob]
A G=[Bob, Harry, Sue] C=[nullset]
A G=[Bob, Harry, Sue] D=[Bob]
A G=[Bob, Harry, Sue] F=[nullset]
D G=[Bob, Charley] A=[Bob]
D G=[Bob, Charley] B=[Bob]
D G=[Bob, Charley] C=[Charley]

D G=[Bob, Charley] F=[nullset]

| | # of | | Column A |
|---|---|---|---|
| | times uniquely | Column A | divided by |
| | identified | divided by | total number of |
| | based on | total number of | intersections |
| | progressive | possible contributors | employed |
| Owner or | intersections | (ignoring the highly | (intersections with E |
| Contributor | (column A) | degenerate list E) | not counted) |
| Bob | 8 | 8/9 = 0.8888 | 8/25 = 0.32 |
| Charley | 3 | 3/9 = 0.3333 | 3/25 = 0.12 |
| David | 0 | 0 | 0 |
| Elvis | 1 | 1/9 = 0.1111 | 1/25 = 0.04 |
| Frank | 1 | 1/9 = 0.1111 | 1/25 = 0.04 |
| Harry | 0 | 0 | 0 |
| Sue | 0 | 0 | 0 |
| Tim | 0 | 0 | 0 |
| Zora | 0 | 0 | 0 |

Comparing this with number of times they are listed as a possible contributor divided by the total number of possible contributors (ignoring the highly degenerate peak, m₅).

| | # of times listed as a possible |
|---|---|
| | contributor divided by the total |
| Owner or Contributor | number of possible contributors |
| Bob | 4/9 = 0.4444 |
| Charley | 3/9 = 0.3333 |
| David | 1/9 = 0.1111 |
| Elvis | 2/9 = 0.2222 |
| Frank | 2/9 = 0.2222 |
| Harry | 2/9 = 0.2222 |
| Sue | 2/9 = 0.2222 |
| Tim | 0 |
| Zora | 0 |

Although this example is not mathematically rigorous, it shows that many schemes can be devised for the use of multiple peaks to increase confidence that a given, putative contributor, of that observed mass is indeed responsible. Different methods put different weight on the observance of more than one peak and either increase or decrease the likelihood of making a false positive or false positive identification. Any of the above permutations or combinations of the multiple fragment masses for use in increasing the identifying power of the catalog are viable implementations for the invention disclosed herein. Any of the above methods or quotients could be normalized to give confidence indices that a given organism is present in the sample. This invention claims the use of any rigorous and well-known statistical methods to handle such datasets and comparisons thereof.

In the idealized predicted spectrum in FIG. 3, peaks widths are atomic (zero dispersion, diffusional, or entropic processes are taking place). In another implementation, and perhaps less arbitrary than the one exemplified above, all calculated in silico mass spectra are given a finite peak width equal to the current resolution limits of the instrument (MALDI-TOF instrument in the preferred embodiment). Besides physical factors, resolution of the instrument is determined by the maximum sample rate of the Time Of Flight (TOF) detector. The calculated masses are derived from time of arrival at a detector (typically a multi-channel plate). For purposes of the disclosed invention, all calculated in silico spectra can be given practical peak-widths within, equal to, or just greater than the current resolution limits of the mass spectrometer. The peaks in this practical, but virtual mass spectrum may also be weighted by calculated occurrence of expected masses. Recall that in the generation of a single RNase T1 fragment catalog, for example, that often times degenerate masses are produced more than once, i.e. AUUUCG may be produced three times by an organism and AUUCUG only once from that same organism. Such masses can be integrally/algebraically weighted by the number of times in which they are contributed etc. so that the observance of a given mass takes on more (or less) meaning. The shape of the calculated peaks may also take on any mathematically advantageous profile. Peaks may be step functions with square shoulders, Dirac-deltas, etc. Regardless of the shape of the virtual or calculated function (or semicontinuous or discontinuous function) it can then be correlated with the observed or experimental mass spectra. Correlation functions, auto-correlation functions, convolutions, Fourier transform analysis or other practical, well-understood prior analysis for comparing data is claimed by the invention. In any putative sample of fragment masses generated by a mixture of organisms, the observed spectra will contain more peaks than any of the controlled fragmentation catalogues generated from a single organism taken alone (unless compositional information for the specie is completely degenerate which the inventors have shown to be highly unlikely unless the specie are closely related). Conceptually, it is beneficial to “overlay” a virtual or calculated mass spectrum over the observed and calculate a correlation coefficient or arbitrary quotient.

Regardless of the mathematical or analytical implementation, once a list or single organism is identified or classified by some confidence, the organism can be placed into phylogenetic context with some or complete accuracy. In one embodiment, “hot-spots” in an existing phylogenetic tree can “light-up” for organisms that are apparently present. In another embodiment or the same, previously unknown organisms can “light-up” the tree proportional to the similarity or related-ness they share with previously known organisms. This would be done by color-maps with intensity or hue proportional to the final index of probability that the particular organism was indeed in the sample. Finally, identification above a certain threshold could call up all known or some subset of known information about the organism, such as known virulence, microscopic images, or any other information deemed interesting in the context of the application, such as for educational purposes.

Depending on the context of the sample, analysis may be greatly simplified. For example, the U.S. Environmental Protection Agency has published on its website a Total Coliform Rule [www.epa.gov] as follows:
“There are a variety of bacteria, parasites, and viruses which can cause immediate (though usually not serious) health problems when humans ingest them in drinking water. Testing water for each of these germs would be difficult and expensive. Instead, water quality and public health workers measure coliform levels. The presence of any coliforms in drinking water suggests that there may be disease-causing agents in the water.
The Total Coliform Rule (published 29 Jun. 1989/effective 31 Dec. 1990) set both health goals (MCLGs) and legal limits (MCLs) for total coliform levels in drinking water. The rule also details the type and frequency of testing that water systems must do.

The coliforms are a broad class of bacteria which live in the digestive tracts of humans and many animals. The presence of coliform bacteria in tap water suggests that the treatment system is not working properly or that there is a problem in the pipes. Among the health problems that contamination can cause are diarrhea, cramps, nausea and vomiting. Together these symptoms comprise a general category known as gastroenteritis. Gastroenteritis is not usually serious for a healthy person, but it can lead to more serious problems for people with weakened immune systems, such as the very young, elderly, or immuno-compromised.
In the rule, EPA set the health goal for total coliforms at zero. Since there have been waterborne disease outbreaks in which researchers have found very low levels of coliforms, any level indicates some health risk.”
   In most cases, to meet the requirements of a broad index such as specified in the Total Coliform Rule, culture-based techniques would be used, although hybridization probes, PCR, or quantitative-PCR, can be employed to obtain more specific and/or quantitative information. Using the invention described herein, a user might design a system concerned with identifying a fairly small subset of uniquely problematic offenders (organisms). As only an example, the system might be designed (with or without nucleic acid amplification) to screen for E. coli, Cryptosporidium, and Giardia simultaneously. The lineages of the three organisms are given below:
E. coli: Bacteria; Proteobacteria; Ganimaproteobacteria; Enterobacteriales; Enterobacteriaceae; Escherichia
Cryptosporidium; Eukaryota; Alveolata; Apicomplexa; Coccidia; Eimeriida; Cryptosporidiidae
Giardia; Eukaryota; Diplononadida group; Diplomonadida; Hexamitidae; Giardiinae

While the latter two are eukaryotes, their small-subunit (ssu) rRNA or 18S rRNA will certainly be compatible with the methods described in this invention. Furthermore, the T1 generated catalogues for each individual organism (or its larger group) will certainly have some number of fragment compositions mutually exclusive to fragments from the others. In the context of this example, any other observed experimental fragment masses not expected from the three organisms could be ignored (but duly noted), and the purposes of the system could be mainly to comply with a governmental or regulatory standard. The concept of ignoring observed compositions can be further extended to background subtraction. An organism of interest could be identified as present among a high, uninteresting background population of another organism by subtracting the background fragments from the spectra. Any fragment masses unique to the minority population (or single cell) would remain. Other examples might include HIV-detection among a high human DNA or RNA background, or pathogen detection among a large background of livestock DNA or RNA. Many other sample-context-situations could be imagined and the invention herein claims specific utility in exploiting such situations.

In another implementation, rRNA or any other characteristic RNA is amplified by reverse transcription (RT) to cDNA or amplified and then forward transcribed back to RNA in a process sometimes referred to as “Eberwine”-like amplification [Van Gelder, R. N., von Zastrow, M. E., Yool, A., Dement, W. C., Barchas, J. D. and Eberwine, J. H., 1990 PNAS USA. 87: 1663-1667 and Eberwine, et al. PNAS. 89: 3010]. During the forward, T7 RNA polymerase-mediated transcription, modified bases may be 100% incorporated, improving the 1 Dalton mass difference between U and C. The resulting amplified, antisense “aRNA” may be used for fragmentation (enzymatic or otherwise). Typically, Eberwine amplification is practiced by joining an oligo-dT primer complimentary to messenger RNAs (especially eukaryotic mRNA) and a T7 RNA polymerase promoter sequence. Modified nucleotides of the final RNA T7 runoff product contain modified nucleotides for fluorescent labeling useful in hybridization microarray experiments. It is beneficial to modify this procedure for mass spectrometric purposes. The T7 promoter sequence can be joined to one or more “Universal” primers [Weisburg, et al. J. of Bacteriology, January 1991, p. 697-703] designed to hybridize to a large portion of all living organisms.

The following sequence is a particularly useful example: 5′-aaa cga cgg cca gtg aat tgt aat acg act cac tat agg cgc AAG GAG GTG ATC CAG CC-3′ The lower case letters are a T7 RNA polymerase promoter sequence. Upper case is universal Weisburg “rd1” primer which recognizes the 3′-end of many bacterial 16S sequences.

The RNA of HIV could be selectively amplified in the same manner. By incorporating only modified bases (especially U or C) in the final runoff transcription, antisense, amplified RNA containing mass-modified bases is created. In addition, the aRNA digestion pattern may be used in conjunction with restriction digest of the intermediate Eberwine reaction product, cDNA, as an independent fragmentation mechanism that results in a mass fragment fingerprint. Tables 1 and 2 compare the restriction fragments of ribosomal DNA (DNA encoding the 16S ribosomal gene) belonging to two bacteria, E. coli and Vibrio Proteolyticus. Tables 1 and 2 are “double-digests” showing the fragments that would be created by treating with two different restriction enzymes that recognize different 4-base recognition sites. Restriction enzymes will often not cut sites located too near the end of a double-stranded DNA substrate, however the fragment calculation algorithm could easily filter the dataset.

TABLE 1

16S rDNA fragments (unsorted) for E. coli generated by
double restriction digest with Alu1 and Dpn1. The lightest
three approximate masses = 7mer 4200; 11mer 6600; 16mer
9600;

AAATTGAAGAGTTTGA
TCATGGCTCAGATTGAACGCTGGCGGCAGGCCTAACACATGCAAGTCGAACGGTAACAGGAAGAAG
CTTGCTTCTTTGCTGACGAGTGGCGGACGGGTGAGTAATGTCTGGGAAACTGCCTGATGGAGGGG
GATAACTACTGGAAACGGTAG
CTAATACCGCATAACGTCGCAAGACCAAAGAGGGGGACCTTCGGGCCTCTTGCCATCGGATGTGC
CCAGATGGGATTAG
CTAGTAGGTGGGGTAACGGCTCACCTAGGCGACGATCCCTAG
CTGGTCTGAGAGGATGACCAGCCACACTGGAACTGAGACACGGTCCAGACTCCTACGGGAGGCAG
CAGTGGGGAATATTGCACAATGGGCGCAAGCCTGATGCAGCCATGCCGCGTGTATGAAGAAGGCC
TTCGGGTTGTAAAGTACTTTCAGCGGGGAGGAAGGGAGTAAAGTTAATACCTTTGCTCATTGACGTT
ACCCGCAGAAGAAGCACCGGCTAACTCCGTGCCAGCAGCCGCGGTAATACGGAGGGTGCAAGCG
TTAATCGGAATTACTGGGCGTAAAGCGCACGCAGGCGGTTTGTTAAGTCAGATGTGAAATCCCCGG
GCTCAACCTGGGAACTGCATCTGATACTGGCAAG
CTTGAGTCTCGTAGAGGGGGGTAGAATTCCAGGTGTAGCGGTGAAATGCGTAGAGA
TCTGGAGGAATACCGGTGGCGAAGGCGGCCCCCTGGACGAAGACTGACGCTCAGGTGCGAAAGC
GTGGGGAGCAAACAGGATTAGATACCCTGGTAGTCCACGCCGTAAACGATGTCGACTTGGAGGTT
GTGCCCTTGAGGCGTGGCTTCCGGAG
CTAACGCGTTAAGTCGACCGCCTGGGGAGTACGGCCGCAAGGTTAAAACTCAAATGAATTGACGG
GGGCCCGCACAAGCGGTGGAGCATGTGGTTTAATTCGATGCAACGCGAAGAACCTTACCTGGTCTT
GACATCCACGGAAGTTTTCAGAATGAGAATGTGCCTTCGGGAACCGTGAGACAGGTGCTGCATGGC
TGTCGTCAG
CTCGTGTTGTGAAATGTTGGGTTAAGTCCCGCAACGAGCGCAAGCCTTATCCTTTGTTGCCAGCGG
TCCGGCCGGGAACTCAAAGGAGACTGCCAGTGATAAACTGGAGGAAGGTGGGGATGACGTCAAGT
CATCATGGCCCTTACGACCAGGGCTACACACGTGCTACAATGGCGCATACAAAGAGAAGCGACCTC
GCGAGAGCAAGCGGACCTCATAAAGTGCGTCGTAGTCCGGATTGGAGTCTGCAACTCGACTCCAT
GAAGTCGGAATCGCTAGTAATCGTGGA
TCAGAATGCCACGGTGAATACGTTCCCGGGCCTTGTACACACCGCCCGTCACACCATGGGAGTGG
GTTGCAAAAGAAGTAGGTAG
CTTAACCTTCGGGAGGGCGCTTACCACTTTGTGATTCATGACTGGGGTGAAGTCGTAACAAGGTAA
CCGTAGGGGAACCTGCGGTTGGATCACCTCCTTA

TABLE 2

16S rDNA fragments (unsorted) for V. proteolyticus
generated by double restriction digest with Alu1 and Dpn1.
The lightest three approximate masses = 7mer 4200; 8mer
4800; 17mer 10,200Da

GAGUUUGA
UCAUGGCUCAGAUUGAACGCUGGCGGCAGGCCUAACACAUGCAAGUCGAGCGGAAACGAGUUAU
CUGAACCUUCGGGGAACGAUAUCGGCGUCGAGCGGCGGACGGGUGAGUAAUGCCUGGGAAAUU
GCCCUGAUGUGGGGGAUAACCAUUGGAAACGAUGGCUAAUACCGCAUAAUAG
CUUCGGCUCAAAGAGGGGGACCUUCGGGCCUCUCGCGUCAGGAUAUGCCCAGGUGGGAUUAG
CUAGUUGGUGAGGUAAGGGCUCACCAAGGCGACGA
UCCCUAG
CUGGUCUGAGAGGAUGA
UCAGCCACACUGGAACUGAGACACGGUCCAGACUCCUACGGGAGGCAGCAGUGGGGAAUAUUG
CACAAUGGGCGCAAGCCUGAUGCAGCCAUGCCGCGUGUGUGAAGAAGGCCUUCGGGUUGUAAA
GCACUUUCAGUCGUGAGGAAGGUAGUGUAGUUAAUAGAUGCAUUAUUUGACGUUAGCGACAGAA
GAAGCACCGGCUAACUCCGUGCCAGCAGCCGCGGUAAUACGGAGGGUGCGAGCGUUAAUCGGA
AUUACUGGGCGUAAAGCGCAUGCAGGUGGUGUGUUAAGUCAGAUGUGAAAGCCCGGGGCUCAA
CCUCGGAAUAGCAUUUGAAACUGGCAGACUAGAGUACUGUAGAGGGGGGUAGAAUUUCAGGUG
UAGCGGUGAAAUGCGUAGAGA
UCUGAAGGAAUACCGGUGGCGAAGGCGGCCCCCUGGACAGAUACUGACACUCAGAUGCGAAAGC
GUGGGGAGCAAACAGGAUUAGAUACCCUGGUAGUCCACGCCGUAAAACGAUGUCUACUUGGAGG
UUGUGGCCUUGAGCCGUGGCUUUCGGAG
CUAACGCGUUAAGUAGACCGCCUGGGGAGUACGGUCGCAAGAUUAAAACUCAAAUGAAUUGACG
GGGGCCCGCACAAGCGGUGGAGCAUGUGGUUUAAUUCGAUGCAACGCGAAGAACCUUACCUAC
UCUUGACAUCCAGAGAACUUUCCAGAGAUGGAUUGGUGCCUUCGGGAACUCUGAGACAGGUGC
UGCAUGGCUGUCGUCAG
CUCGUGUUGUGAAAUGUUGGGUUAAGUCCCGCAACGAGCGCAACCCUUAUCCUUGUUUGCCAG
CACGUAAUGGUGGGAACUCCAGGGAGACUGCCGGUGAUAAACCGGAGGAAGGUGGGGACGACG
UCAAGUCAUCAUGGCCCUUACGAGUAGGGCUACACACGUGCUACAAUGGCGCAUACAGAGGGCG
GCCAACUUGCGAAAGUGAGCGAAUCCCAAAAAGUGCGUCGUAGUCCGGAUUGGAGUCUGCAACU
CGACUCCAUGAAGUCGGAAUCGCUAGUAAUCGUGGA
UCAGAAUGCCACGGUGAAUACGUUCCCGGGCCUUGUACACACCGCCCGUCACACCAUGGGAGU
GGGCUGCAAAAGAAGUGGGUAGUUUAACCUUCGGGAGGACGC

In this implementation, some portion of the cDNA containing a T7 RNA polymerase promoter would be sacrificed for restriction digest and fragments would be observed in the MALDI. The rest of the cDNA would go on to be transcribed in the Eberwine process and then treated with endoribonuclease to create an independent mass fragmentation pattern. The ability to unambiguously assign monomer composition goes down as the length of a fragment increases, so any restriction digest would have to generate an identifying pattern of masses of light enough molecular weight to assign composition accurately and transfer to the gas phase efficiently if the mass spectrometry method is MALDI, ESI, or any other “soft” ionization technique. As instrument design and experimental techniques improve, this low-pass filtering effect on mass will improve.

One challenge to analyzing nucleic acid fragments using MALDI-TOF mass spectrometry is the appearance of “daughter” peaks mainly introduced by cation adducts bound to the polyphosphate backbone of DNA or RNA. These daughter peaks can sometimes obscure isotopic information or other nearby fragment masses in complex mixtures. This problem can be largely solved by those skilled in the art by proper sample preparation techniques, such as reverse-phase purification using hydrophobic C-18 columns, ZipTips®, a commercial product offered by Millipore, desalting columns, size-exclusion buffer exchange gels or columns, mixed-bed ion exchangers, or proper buffer selection (ammonium salts are preferred). Any process, however, that would allow incorporation of a non-charged backbone would increase the simplicity and analysis of the mass spectra. For example peptide nucleic acids have an uncharged, amide-bond backbone. Either during amplification or replication of the ICM, or after fragments are generated, if bases can be incorporated with uncharged backbone elements, spectrum quality would improve. An endoribonuclease such as RNase T1 would be dependent upon the phosphate bond at the 3′-end of G and the 2′-OH of that same G residue, however all other nucleotides could have a peptide linkage. The resulting fragments or the ICM starting material would be a hybrid molecule with readily (and specifically) hydrolysable bonds after G residues, and an uncharged backbone elsewhere. Similarly, if an RNA or DNA can be replicated into PNA containing the same sequence information, the PNA-ICM could be fragmented in a base-specific manner by engineered enzymes. SELEX or In vitro selection methods, or directed evolution methods known to those skilled in the art make it highly feasible that an enzyme could be developed, engineered, or isolated from nature that could fragment peptide nucleic acids in a controllable or base-specific manner. In a preferred embodiment, one may use of any such enzyme for use in producing nucleic acid analog fragments with uncharged backbones, thereby improving the quality of the mass spectra. Also claimed is the use of any restriction enzyme identified that has acceptable activity for restriction of a PNA sequence, leading to a characteristic fragment pattern in a mass spectrometer.

Treatment of RNA with base-specific ribonucleases is well known in the field. The present invention encompasses any method that results in a controlled and known fragmentation pattern that can be simulated by computer. Signature oligonucleotides can be produced by digesting the characteristic molecule with ribonuclease T1, ribonuclease A, ribonuclease PhyM, ribonuclease U2 or any other base specific endoribonuclease or chemical reagent.

In an alternative embodiment, the characteristic Information Containing Molecule, might not be a nucleic acid. Proteins and subfragments thereof might contain signature quality characteristic of a given organism, group of organisms, or disease state. As long as fragments could be produced in a reproducible manner, these characteristic compositions could be catalogued using the same approach that has been employed with small subunit ribosomal RNA.

In one embodiment, the system will obtain a nucleic acid in any quantity sufficient for the detection limits of the mass spectrometer. Ribosomal RNA, for example, may be isolated from tissue or cell culture either from a mixture of organisms or from an appropriately treated soil sample. Separation of the nucleic acid molecule of interest, i.e. 5S, 16S, or 23S rRNA, rDNA, etc. prior to enzymatic treatment may be accomplished by any suitable adsorptive, precipitation or affinity method. This separation may take place in parallel such as in a 96-well format. 96 capillaries, for example may electrophorese sample directly to a MALDI-TOF plate where enzymatic treatment occurs prior to mass-spectrometric analysis. Each well may contain a mixture of rRNA molecules from different organisms or may contain the rRNA from a culture of a single organism. Peaks present in the mass spectrum (spectra) are then compared with in silico digests of sequences obtained from any suitable database of rRNA sequences. Separation or purification of the ICM may not be necessary. Calculations can be performed to determine if too much information would be lost (too many degenerate compositions) by treating total RNA with the fragmentation method, e.g. ribonuclease T1 digestion. In other words, calculations can be performed to include 5S and 23S or other “contaminating” RNA as part of the ICM starting material, to see if identifying power decreases or possibly increases. Alternatively the ICM of interest may be selectively enriched-for or amplified above other contaminants. Fragments subsequently generated would be the dominant products and any contaminating sequences (compositions) would remain obscured in the baseline noise of the mass spectrometer.

Many, integrated “front-end” systems for preparing the ICM of interest could be conceived. Automated lab-on-a-chip type devices for combining any amplification steps or the enzymatic digestion or fragmentation could be implemented. Chromatographic steps could be automated so that only the ICM of interest is fragmented and/or deposited on the input device (spotted on the MALDI plate in the preferred embodiment). Other sample preparation steps may be automated in this fashion or by robots or spotters. This invention claims that any of these automation procedures are beneficial and may be part of the system.

As a demonstration of the informatics portion of the system, 16S rRNA sequences were taken from 7,322 prokaryotic organisms obtained from Ribosomal Database Project (RDP) Release 7.1. 1,921 of the sequences met minimum criteria for sequence sufficiency. Table 1 shows the results of in silico enzymatic digestion of 16S rRNA sequences from the corresponding 1,921 organisms. Two conditions for the digest were inherently assumed:
The 16S rRNAs from these organisms are intact and free of contaminating rRNA.
All of the endoribonuclease digestions of 16S rRNAs are complete (no internal G residues remain).

The following program, “Catalog.pl” written in Perl generates an RNase T1 or RNase A catalogue of input sequences:

```
#!/usr/local/bin/perl -w
# ./catalogue
# This program parses the phylogenetic tree in newick format.
use strict;
use DBI;
use Storable;
use constant U => 305.17;
use constant G => 344.23;
use constant C => 304.20;
use constant A => 328.26;
use constant H => 1;
use constant PO4 => 94.97;
use constant OH => 17;
my (%TlcatalogueTable, %AcatalogueTable);
my (@sequenceArray); # the 16S seq. arrays used for RNase T1 and A.
my ($org, $cat, $freq, $length, $mw);
my $reply;
open(SEQ_FILE, “SSU_Prok.fasta.flat.valid”) or die “Cannot open the file:
$?”;
#open(SEQ_FILE, “test”) or die “Cannot open the file.”;
foreach (<SEQ_FILE>)
  {
    chomp;
    m/\hat{ }(.+)\t(.+)/;
    @sequenceArray = split(//, $2);
    $T1catalogueTable{$1} = { }; # the value is a reference to an anonymous
hash.
    catalog(‘RNase T1’, \@sequenceArray, $T1catalogueTable{$1});
    $AcatalogueTable{$1} = { }; # the value is a reference to an anonymous
hash.
    catalog(‘RNase A’, \@sequenceArray, $AcatalogueTable{$1});
  }
close SEQ_FILE;
store(\%T1catalogueTable, ‘T1catalogueTable.bin’);
store(\%AcatalogueTable, ‘AcatalogueTable.bin’);
buildHash(‘RNase T1’);
buildHash(‘RNase A’);
#printTable(‘RNase T1’);
#printTable(‘RNase A’);
print “The old data in the database Catalogue16S will be flushed. Continue?
”;
chomp($reply = <STDIN>);
if ($reply =˜ m/y/)
  {
    print “This may take some time ...\n”;
    add2database(‘RNase T1’);
    add2database(‘RNase A’);
  }
#######
sub catalog
  {
    my ($enzyme, $arrayRef, $hashRef) = @_;
    my $counter = 1;
    my @temp;
    my $catalogue;
    foreach (@$arrayRef)
      {
      push(@temp, $_);
      if (($enzyme eq ‘RNase T1’ and ($_{—} eq ‘G’ or $_{—} eq ‘g’)) # RNase T1.
        or
        ($enzyme eq ‘RNase A’ and ($_{—} eq ‘U’ or $_{—} eq ‘u’ or $_{—} eq ‘C’ or
$_{—} eq ‘c’))) # RNase A.
      {
        $catalogue = join(‘’, @temp);
        if ($counter == @temp) # This oligo happens at the 5′ end of this
16S
          {
          $catalogue = ‘(P ) - ’ . $catalogue . ‘ - (P )’;
          }
        elsif ($counter == @$arrayRef) # This oligo happens at the 3′ end
of this 16S
          {
          $catalogue = ‘(OH) - ’ . $catalogue . ‘ - (OH)’;
          }
        else # This oligo happens in the middle of this 16S
          {
          $catalogue = ‘(OH) - ’ . $catalogue . ‘ - (P )’;
          }
        if (not exists $hashRef->{$catalogue}) # this catalogue appears
for the first time.
          {
```

| | |
|---|---|
| $hashRef->{$catalogue} = [ ]; | # the value is a reference to an |
| anonymous array. | |
| | # 1st [0] element records where it |
| appear: | |
| | # 5′ end(1), the middle(2), |
| or 3′ end(3) | |
| | # 2nd [1] element is the appearing |
| frequency in this 16S | |
| | # 3rd [2] element is the length |
| | # 4th [3] element is the |

| | |
|---|---|
| molecular weight. | |
| if ($counter == @temp) | |
| { | |
| $hashRef->{$catalogue}[0] = 1; | # This oligo happens at the |
| 5′ end of this 16S | |
| } | |
| elsif ($counter == @$arrayRef) | |
| { | |
| $hashRef->{$catalogue}[0] = 3; | # This oligo happens at the |
| 3′ end of this 16S | |
| } | |
| else | |
| { | |
| $hashRef->{$catalogue}[0] = 2; | # This oligo happens in the |

```
middle of this 16S
          }
        $hashRef->{$catalogue}[1] = 1; # set the number of this cat. to
1.
        $hashRef->{$catalogue}[2] = scalar @temp;
        foreach my $nt (@temp)
          {
```

```
if ($nt eq ‘U’ or $nt eq ‘u’)
  {
  $hashRef->{$catalogue}[3] += U;
  }
if ($nt eq ‘G’ or $nt eq ‘g’)
  {
  $hashRef->{$catalogue}[3] += G;
  }
if ($nt eq ‘C’ or $nt eq ‘c’)
  {
  $hashRef->{$catalogue}[3] += C;
  }
if ($nt eq ‘A’ or $nt eq ‘a’)
  {
  $hashRef->{$catalogue}[3] += A;
  }
```

```
  }
if ($hashRef->{$catalogue}[0] == 1)
  {
    $hashRef->{$catalogue}[3] += PO4;
    $hashRef->{$catalogue}[3] += H;
  }
elsif ($hashRef->{$catalogue}[0] == 2)
  {
    $hashRef->{$catalogue}[3] += OH;
    #$hashRef->{$catalogue}[3] += H;
  }
else
  {
    $hashRef->{$catalogue}[3] += OH;
    $hashRef->{$catalogue}[3] += OH;
    $hashRef->{$catalogue}[3] −= PO4;
    }
  }
else # increment the number if it reappears.
  {
  $hashRef->{$catalogue}[1]++;
  }
@temp = ( );
```

```
        }
    $counter++;
    }
  # The following is for the last catalogue in the sequence if it does not
end in ‘G|g’.
  if (@temp >= 1)
    {
    $catalogue = join(‘’, @temp);
    $catalogue = ‘(OH) - ’ . $catalogue . ‘ - (OH)’;
```

| | |
|---|---|
| if (not exists $hashRef->{$catalogue}) | # this catalogue appears for |
| the first time. | |
| { | |
| $hashRef->{$catalogue} = [ ]; | # the value is a reference to an |
| anonymous array. | |
| $hashRef->{$catalogue}[0] = 3; | # This oligo ALWAYS happens at the |
| 3′ end of this 16S | |
| $hashRef->{$catalogue}[1] = 1; | # set the number of this cat. to |

```
1.
        $hashRef->{$catalogue}[2] = scalar @temp;
        foreach my $nt (@temp)
```

```
{
if ($nt eq ‘U’ or $nt eq ‘u’)
  {
    $hashRef->{$catalogue}[3] += U;
  }
if ($nt eq ‘G’ or $nt eq ‘g’)
  {
    $hashRef->{$catalogue}[3] += G;
  }
if ($nt eq ‘C’ or $nt eq ‘c’)
  {
    $hashRef->{$catalogue}[3] += C;
  }
if ($nt eq ‘A’ or $nt eq ‘a’)
  {
    $hashRef->{$catalogue}[3] += A;
  }
}
```

```
          $hashRef->{$catalogue}[3] += OH;
          $hashRef->{$catalogue}[3] += OH;
          $hashRef->{$catalogue}[3] −= PO4;
        }
      else # increment the number if it reappears.
        {
          $hashRef->{$catalogue}[1]++;
        }
      @temp = ( );
    }
  }
#########
sub buildHash
  {
    my ($enzyme) = @_;
    my (%catalogueTable, $mr2orgFileName, $org2mrFileName);
    my ($org, $oligo);
    my (%mr2org, %org2mr);
    if ($enzyme eq ‘RNase T1’)
      {
      %catalogueTable = %TlcatalogueTable;
      $mr2orgFileName = ‘Tlmr2org.bin’;
      $org2mrFileName = ‘Tlorg2mr.bin’;
      }
    if ($enzyme eq ‘RNase A’)
      {
      %catalogueTable = %AcatalogueTable;
      $mr2orgFileName = ‘Amr2org.bin’;
      $org2mrFileName = ‘Aorg2mr.bin’;
      }
    foreach $org (keys %catalogueTable)
      {
      $org2mr{$org} = { };
      foreach $oligo (keys %{$catalogueTable{$org}})
        {
          $org2mr{$org}{$catalogueTable{$org}{$oligo}[3]} = undef;
          $mr2org{$catalogueTable{$org}{$oligo}[3]} = { } if(not exists
$mr2org{$catalogueTable{$org}{$oligo}[3]});
          $mr2org{$catalogueTable{$org}{$oligo}[3]}{$org} = undef;
        }
      }
    store(\%mr2org, $mr2orgFileName);
    store(\%org2mr, $org2mrFileName);
  }
#########
sub printTable
  {
    my ($enzyme) = @_;
    my %table;
    my ($catalogue, $orgName);
    my @tempTable;
    if ($enzyme eq ‘RNase T1’)
      {
%table = %TlcatalogueTable;
      }
    if ($enzyme eq ‘RNase A’)
      {
%table = %AcatalogueTable;
      }
    print “\n\n$enzyme digestion:\n\n”;
    print “Organism Oligo
Freq. Leng. Mr\n”;
    print “------------------------------------------------------------------
--------------------\n\n”;
    # Output is sought by organism names.
    foreach $orgName (sort {$a cmp $b} keys %table)
      {
      print “$orgName\n”;
      foreach $catalogue (sort { $table{$orgName}{$b}[2] <=>
$table{$orgName}{$a}[2]
```

∥

$a cmp $b }

keys %{$table{$orgName}})

```
        {
          push @tempTable, [$orgName, $catalogue,
$table{$orgName}{$catalogue}[1], $table{$orgName}{$catalogue}[2],
$table{$orgName}{$catalogue}[3]];
          if ($table{$orgName}{$catalogue}[2] >= 12)
```

{
$cat = $catalogue;
$cat =˜ s/\(OH\) - / /;
$cat =˜ s/ - \(p \)/ /;
$freq = $table{$orgName}{$catalogue}[1];
$length = $table{$orgName}{$catalogue}[2];
$mw = $table{$orgName}{$catalogue}[3];
$˜ = ‘SORTBYORG’;
write (STDOUT);
}

```
          }
        print “\n”;
        }
      print “\n\n$enzyme digestion:\n\n”;
      print “Organism Oligo
Freq. Leng. Mr\n”;
      print “------------------------------------------------------------------
      --------------------\n\n”;
      # Output is sought by the oligo sizes
      foreach (sort {$b->[3] <=> $a->[3] ∥ $a->[1] cmp $b->[1]} @tempTable)
        {
        if ($_->[3] >= 12)
          {
```

$org = $_->[0];
$cat = $_->[1];
$cat =˜ s/\(OH\) - / /;
$cat =˜ s/ - \(P \)/ /;
$freq = $_->[2];
$length = $_->[3];
$mw = $_->[4];
$˜ = ‘SORTBYSIZE’;
write (STDOUT);

```
          }
        }
      print “\n”;
    }
#######
format SORTBYORG =
```

@<<<<<<<<<<<<<<<<<<<<<<<<<<<<<<<<<<<<<<<<<<<<<<<<<<<< @<<<< @<<<<

@####.##

| | |
|---|---|
| $cat, | $freq, |

$length, $mw
.
#######
format SORTBYSIZE =
@<<<<<<<<< @<<<<<<<<<<<<<<<<<<<<<<<<<<<<<<<<<<<<<<<<<<<<<<<<<<<< @<<<< @<<<<
@####.##

| | | |
|---|---|---|
| $org, | $cat, | $freq, $length, |

```
$mw
.
#######
sub add2database
  {
    my ($enzyme) = @_;
    my (%table, $databaseTableName, $dbInputFile);
    my ($catalogue, $orgName);
    my ($dbh, $sth);
    if ($enzyme eq ‘RNase T1’)
      {
      %table = %TlcatalogueTable;
      $databaseTableName = ‘catalogueByTl’;
      $dbInputFile = ‘catalogueByTl.txt’;
      }
    if ($enzyme eq ‘RNase A’)
      {
      %table = %AcatalogueTable;
      $databaseTableName = ‘catalogueByA’;
      $dbInputFile = ‘catalogueByA.txt’;
      }
    $dbh = DBI->connect(‘DBI:mysql:Catalogue16S:localhost’, ‘httpd’, undef)
        or die “cannot connect to Catalogue16S: $DBI::errstr”;
    $dbh->do(“delete from $databaseTableName”);
    open(OUT, “>$dbInputFile”);
    foreach $orgName (keys %table)
      {
      foreach $catalogue (keys %{$table{$orgName}})
        {
          #$dbh->do(“insert $databaseTableName (organismName, oligo,
frequency, length, molecularWeight)”
      # . “values (‘$orgName’, ‘$catalogue’,
‘$table{$orgName}{$catalogue}[1]’, ‘$table{$orgName}{$catalogue}[2]’,
‘$table{$orgName}{$catalogue}[3]’)”);
          print OUT
“$orgName\t$catalogue\t$table{$orgName}{$catalogue}[1]\t$table{$orgName}{$catalogue}
[2]\t$table{$orgName}{$catalogue}[3]\n”;
        }
      }
    close(OUT);
    $dbh->do(“load data infile ‘/home/zzhang/16S_catalogue/$dbInputFile’ into
table $databaseTableName”);
    $dbh->disconnect( );
  }
```

Digestion by the endoribonuclease, RNase T1 yields a greater number of distinct masses for any given organism than ribonuclease A. RNase T1 also yielded a greater number of masses capable as acting as unique identifiers for a single organism. 221 (11.5%) of the 1,921 bacteria under consideration could be uniquely identified by the molecular weight of a single unique oligonucleotide in their RNase T1-digested 16S rRNA.

TABLE 3
The distribution of the various n-mers produced by endoribonuclease
digestion at the time “Catalog.pl” was executed for 1,921 valid input
sequences, where n is the number of nucleotides in the fragment.

| | Attributes of the oligonucleotide catalogue | | | | | |
|---|---|---|---|---|---|---|
| Ribonuclease | Rₗ | N_{a.o.} | N_{d.o.} | N_{d.Mr.} | A_{bar}_o | A_{bar}_Mr |
| RNase T1 | 2-54 | 246,125 | 8,928 | 1,077 | 130 | 79 |
| RNase A | 2-21 | 154,613 | 2,129 | 325 | 84 | 54 |

Rl—Length range
Na.o.—Number of all oligonucleotides
Nd.o.—Number of distinct oligonucleotides
Nd.Mr.—Number of distinct molecular weights
Abar_o—Average number of distinct oligonucleotides that a 16S rRNA digested by endoribonuclease will produce.
Abar_Mr—Average number of different molecular weights of oligonucleotides that a 16S rRNA digested by endoribonuclease will produce.

While only 11.5% of the filtered set of 1,921 organisms were uniquely identifiable by the presence of a single oligonucleotide composition (mass), any real environmental sample will likely contain a much smaller subset of organisms. In the preferred embodiment of the invention, numerous statistical techniques may be employed to increase confidence in the identification of an organism based on the simultaneous presence of multiple characteristic masses, especially when those masses are known to be mutually exclusive to another organism appearing in the sample. With no direct chemical modification or incorporation of modified bases, for RNA digests, the best discriminating power of the system requires resolution of approximately 1 Dalton, the mass difference between Uridine and Cytidine. For restriction endonuclease digests of rDNA, the resolution requirements relax as the nearest-neighbor nucleotides in mass are deoxythimidine and deoxyadenosine (a difference of approx. 9.013Da). In terms of resolution, however, RNA is preferred over double-stranded in that the same sequence information is present in less overall mass.

While the invention preferably utilizes software to identify characteristic compositions, it is well known in the art how to program for this purpose. Although the present invention has been disclosed using programs written in Perl and MATLAB, any suitable programming languages and algorithmic approaches may be used to achieve the desired result. All that is required is that a catalogue of fragments is generated and the source organism of the Information Containing Molecule from the sequence database is tracked. An example code for generating T1 fragments from a single input sequence is shown previously in this description.

An additional enzymatic approach for the release of signature sequences may be afforded by the use of an amplification step (polymerase chain reaction or its alternatives) to produce a cDNA corresponding to a region of the rRNA gene rich in signature sequences representing the organisms that are of most relevant to a particular application. The signature sequences might then be released by converting the region back to RNA by the use of T7 runoff transcription followed by ribonuclease digestion. This offers the additional advantage that the T7 polymerase will in some cases be able to insert mass modified bases (e.g. ribothymidine, isotopically labeled bases, amino-allyl U, amino-allyl C, etc.) thereby improving the mass distinctions. Table 3 is a non-exhaustive list for example only of modified nucleotides.

TABLE 4
Non-exhaustive example of commercially available modified nucleotides
for improved mass distinction (Ambion, Inc.)

| Cat# | Product Name | Size |
|---|---|---|
| 8400 | 2′ F-CTP | 10 mM (25 μl) |
| 8402 | 2′ F-UTP | 10 mM (25 μl) |
| 8404 | 2′ NH2-CTP | 10 mM (25 μl) |
| 8405 | 2′ NH2-CTP | 50 mM (50 μl) |
| 8406 | 2′ NH2-UTP | 10 mM (25 μl) |
| 8407 | 2′ NH2-UTP | 50 mM (50 μl) |
| 8416 | 4-thio UTP | 10 mM (25 μl) |
| 8417 | 4-thio UTP | 50 mM (50 μl) |
| 8418 | 5-iodo CTP | 10 mM (25 μl) |
| 8419 | 5-iodo CTP | 50 mM (50 μl) |
| 8420 | 5-iodo UTP | 10 mM (25 μl) |
| 8421 | 5-iodo UTP | 50 mM (50 μl) |
| 8422 | 5-bromo UTP | 10 mM (25 μl) |
| 8426 | Adenosine-5′-(1-thiotriphosphate) | 10 mM (25 μl) |
| 8427 | Adenosine-5′-(1-thiotriphosphate) | 50 mM (50 μl) |
| 8428 | Cytidine-5′-(1-thiotriphosphate) | 10 mM (25 μl) |
| 8429 | Cytidine-5′-(1-thiotriphosphate) | 50 mM (50 μl) |
| 8430 | Guanosine-5′-(1-thiotriphosphate) | 10 mM (25 μl) |
| 8432 | Uridine-5′-(1-thiotriphosphate) | 10 mM (25 μl) |
| 8434 | Pseudo-UTP | 10 mM (25 μl) |
| 8435 | Pseudo-UTP | 50 mM (50 μl) |
| 8436 | 5-(3-aminoallyl)-UTP | 10 mM (25 μl) |
| 8437 | 5-(3-aminoallyl)-UTP | 50 mM (50 μl) |
| 8438 | 5-(3-aminoallyl)-dUTP | 10 mM (25 μl) |
| 8439 | 5-(3-aminoallyl)-dUTP | 50 mM (50 μl) |
| 8440 | Inosine triphosphate | 50 mM (50 μl) |
| 8443 | 7-Deaza-GTP | 10 mM (25 μl) |

Other methods besides mass spectrometry could be employed for determining the overall composition of the generated fragments. Optical properties such as absorbance, fluorescence, or stereochemical properties could be employed for determining composition, especially if modified bases are introduced by enzymatic incorporation or chemical treatment. Circular dichroism, spectrophotometry, or surface plasmon resonance, could serve as feasible methods of measuring fragment composition. Modified compositions could be selected for or enriched by technologies such as immobilized metal affinity chromatography or “IMAC”. For example certain identifying sequences could be selectively modified to contain “handles” which enhance binding to IMAC matrices. Hexa- or poly-histidine tags could be incorporated or added to compositions of interest for enrichment or selection purposes.

Other options for releasing signature sequences might include the use of deoxyribozymes comprising catalytic sequences of DNA which selectively cleave RNASeveral RNA-cleaving deoxyribozyme catalytic motifs have been discovered by in vitro selection or SELEX. One or more 10-23 deoxyribozymes or similar catalytic DNAs can be designed to selectively cut out a region of a larger rRNA molecule. Either conserved or highly variable regions of 16S rRNA, for example, may be excised. The specificity of the substrate-binding arms 1 and 11 and release of any signature sequence in between two target regions would lend great confidence to the presence of a given organism in a mixture. A deoxyribozyme “cocktail” for the release of very many signature sequences, and thus, identification of very many different organisms could be easily designed. Furthermore, the sequence specificity of deoxyribozymes makes it possible to enzymatically treat total ribosomal RNA without purification of a characteristic molecule, i.e. 16S rRNA. While the deoxyribozyme approach may lack somewhat in generality due to the necessity for hybridization, portions of ICM starting material released by deoxyribozymes might contain highly variable or conservative regions that would result in characteristic compositions being released. Additionally, specific compositional inserts in ribosomal RNA could be specifically excised by one or more deoxyribozyme [Pitulle, C, Hedenstierna, KOF, Fox, G E “Artificial Stable RNAs: A Novel Approach for Monitoring Genetically Engineered Microorganisms,” Appl. Env. Micro. 1995; 61: 3661-3666 (1995)]. Such uniquely identifying inserts need not be excised by only deoxyribozymes. The incorporation of “mass-tags” is completely compatible with endoribonuclease digestion as described previously. Detection of such uniquely identifying inserts would be beneficial to the invention, especially if such inserts also contained purification or enrichment “handles” as described herein.

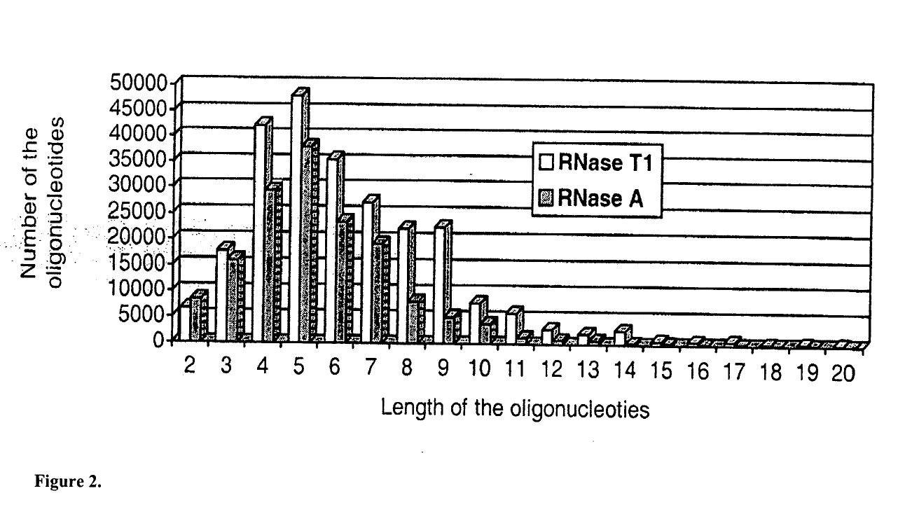

Composition versus sequence. While modified bases may on occasion be present in both DNA and RNA, the number of different sequences using only a four letter alphabet (A,C,G,T or A,C,G,U for DNA or RNA respectively) increases as 4ⁿ where n is the number of bases in the sequence. The number of different mass compositions is always less as determined by the following permutation formula (actually, a combination with replacement):
No. of compositions=(n+3)!/(n!×3!)
where ! denotes factorial. For instance, the number of unique compositions for the complete set of possible 10mers is 13!/(10!×3!) or 286. This is much less than the 410=1,048,576 unique sequences. Unequivocal determination of composition based on mass alone is determined by the resolution of the mass spectrometer. For MALDI-TOF mass spectrometry, operation in linear mode with no internal standards added to the sample is generally considered a “low resolution” technique, typically yielding resolution of m/m of 500-1000 [Null A P, Muddiman D C. J. Mass. Spectrometry. 2001; 36:589]. The mass differences (in ppm) of neighboring compositions can be calculated according to the following formula:
ppm mass difference=[(M₂−M₁)/M₂]×10⁶
Letting M₂=5000Da (roughly a 16mer weight) a resolution of M₂/m of 1000 taken at full-width-half-maximum (FWHM) means that m=5Da. This corresponds to a ppm mass difference of 100, or in other words, only nearest neighbor species of ppm difference greater than 100 would be distinguished at this resolution. Koomen J M, Russell W K, Tichey S E, Russell D H. J. Mass Spectrometry. 2002; 37: 357-371 have published an extensive review of the resolution requirements for accurately determining oligonucleotide composition. They determined that all compositions of DNA of up to 13mers could be accurately assigned at 5 ppm mass accuracy or less. This accuracy is achievable in current MALDI-TOF spectrometers by operating in reflectron mode, employing proper sample preparation techniques, and including internal calibration standards in the sample. In addition, mass distinction can be improved in some embodiments by incorporating non-standard bases and/or isotopically labeled bases into samples. This invention requires no constraints on the mode of operation of the mass spectrometer so long as adequate resolution and sensitivity are achieved.
MALDI-TOF Data of RNA digests. Various researchers have demonstrated that MALDI-TOF spectra of 5S and 16S rRNA digests can be obtained with varying success. Kirpekar, F, Douthwaite, S, Roepstorff, P. RNA. 2000; 6: 296-306 have shown that all expected RNase T1 fragments can be successfully observed in a MALDI spectrum of the 120 nucleotide 5S rRNA molecule See FIG. 2, which shows a calculated distribution of oligonucleotides according to the their lengths from a population of 1,921 organisms generated by RNase T1 and RNase A digestion of 16S rRNA).

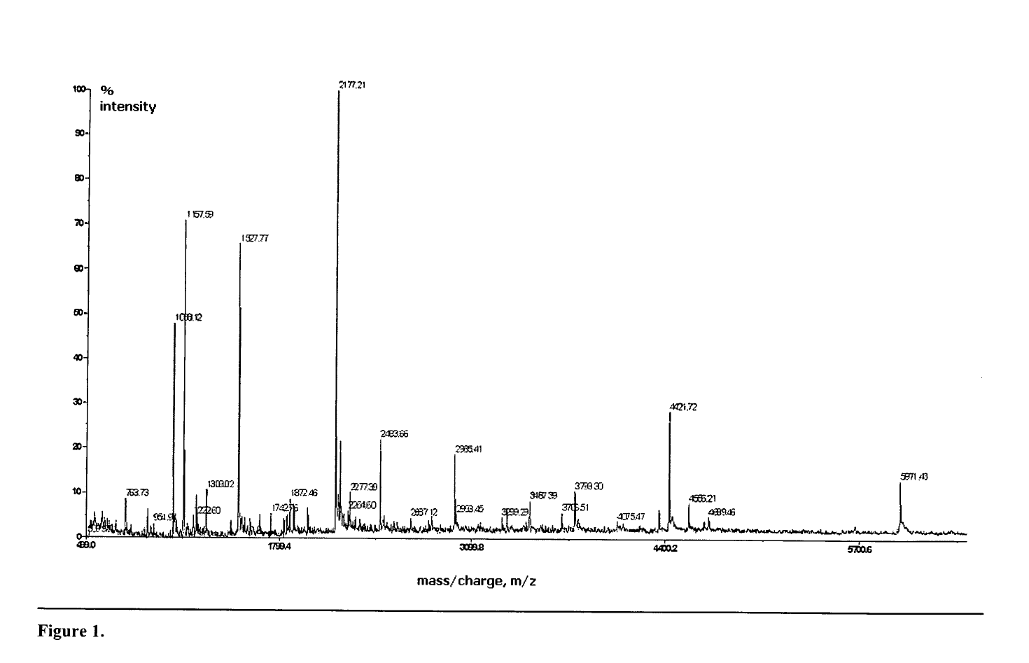

Table 5 along with FIG. 1 show the effectiveness of internal calibration in achieving 1 Da resolution. FIG. 1 shows a Matrix Assisted Laser Desorption Ionization Time of Flight, or MALDI-TOF spectrum of a T1 ribonuclease digest of synthetic 19mer RNA oligonucleotide. The x-axis or abscissa is a measure of mass, in this case mass over charge state of the fragment observed, m/z. The y-axis or ordinate is a normalized intensity of counts of arrival at a Time Of Flight (TOF) detector. The figure is representative of the spectrum resulting from a relatively short starting material in generating a measured fragmentation from said starting material. Other publications generally related to the problem solved by the current invention are:
Hartmer, et al. Nucleic Acids Research. 2003; 31: e47.
Krebs, et al. Nucleic Acids Research. 2003; 31: e37.

Bocker, S. Bioinformatics, Vol. 19 Suppl. 1 2003, pages i44-i53

TABLE 5

Successful measurement of expected masses in a RNase
T1 digest of a 19mer synthetic oligonucleotide.
These data correspond to the experimental mass
spectrum illustrated in FIG. 1.

19mer starting material
5′-CCCCUUG/AUAG/CCG/CUACG-3′

Expected

| | | |
|---|---|---|
| m/z | meas. after | Difference |

| | | | |
|---|---|---|---|
| Sequence (5′-3′) | [M-H-] | calibration | (Da) |

| | | | |
|---|---|---|---|
| CCCCUUG/AUAG/CCG/CUACG-oh | 5971.63 | 5971.48 | 0.15 |
| CCG > p | 954.57 | 954.97 | −0.4 |
| CCCC-oh* | 1157.59 | 1157.59* | 0 |
| AUAG > p | 1308.79 | 1309.02 | −0.23 |
| CUACG-oh | 1527.99 | 1527.77 | 0.22 |
| CGCUUG > p | 2177.27 | 2177.21 | 0.06 |
| CCG/CUACG-oh | 2483.47 | 2483.66 | −0.19 |
| CCCCUUG/AUAG > p | 3487.07 | 3487.39 | −0.32 |
| AUAG/CCG/CUACG-oh | 3793.30 | 3793.30 | 0 |
| 14mer* | 4421.73 | 4421.73* | 0 |

*internal calibrant

Simulation of microbial identification by MALDI-TOF mass spectrometry. A computer simulation was employed to test the effectiveness of the microbial identification method that uses the endoribonuclease-generated signature sequences of 16S rRNA whose molecular weights can be identified by MALDI-TOF mass spectrometry. In addition to the previously listed two assumptions, this program also assumes there is no loss of digestion product in the mass spectrometry experiment.

To simulate the process, this program first randomly selects a number of organisms from the set of 1,921 prokaryotes whose 16S rRNA sequences have been completely sequenced. The 16S rRNAs of these selected organisms are then treated with an endoribonuclease (RNase T1 or RNase A) and as a result a pool of different oligonucleotides is generated.

Example Program “Simulate”. Description of the program is disclosed herein.

```
#!/usr/local/bin/perl -w
# ./simulate
#
use strict;
use Storable;
use constant WIDTH => 0.95;
my ($enzyme) = @ARGV;
my $width = 0;
my (%mr2org, %org2mr);
my ($mr, $org, $prob, $response, $numOfPeaksOnChart, $numOfPeaks);
my ($orgInSample, %orgsInSample, %mrChart, %possibleOrgs); # sets
my ($i, $j, @mrs);
if (@ARGV == 0)
  {
    print “Usage: ./simulate enzyme\n”;
    exit;
  }
elsif ($enzyme eq ‘T1’)
  {
    print “retrieving data ...\n”;
    %mr2org = %{ retrieve(‘T1mr2org.bin’) };
    %org2mr = %{ retrieve(‘T1org2mr.bin’) };
  }
elsif ($enzyme eq ‘A’)
  {
    print “retrieving data ...\n”;
    %mr2org = %{ retrieve(‘Amr2org.bin’) };
    %org2mr = %{ retrieve(‘Aorg2mr.bin’) };
  }
else
  {
    print “Unknown RNase.\n”;
    exit;
  }
while(1)
  {
    print “\nReturn or type ‘exit’ to quit: ”;
    chomp($response = <STDIN>);
    if ($response eq ‘exit’)
      {
      exit;
      }
    else
      {
      $width = $response unless ($response eq ‘’);
      my $randOrgNum = rand(10) + 1;
      my @orgs = keys %org2mr;
      # randomly select some organisms as the samples.
      foreach (1 .. $randOrgNum)
        {
          $orgsInSample{ $orgs[ rand @orgs ] } = undef;
        }
      # generate the Mr peaks in the MS chart.
      foreach $orgInSample (keys %orgsInSample)
        {
          foreach $mr (keys %{$org2mr{$orgInSample}})
            {
            $mrChart{$mr} = ‘valid’; # set the initial value to ‘valid’
            }
        }
      @mrs = sort{$a <=> $b} keys %mrChart;
      for ($i = 0; $i <= $#mrs; $i++)
        {
          for ($j = $i+1; $j <= $#mrs; $j++)
            {
            # if this two peaks are too close (less than the resolution),
            # both of them are marked invalid.
            if ($mrs[$j] − $mrs[$i] < $width)
              {
                  $mrChart{$mrs[$j]} = ‘invalid’;
                  $mrChart{$mrs[$i]} = ‘invalid’;
              }
            }
        }
      # generate the collection of all possible organisms from all peaks.
      foreach $mr (keys %mrChart)
        {
          if ($mrChart{$mr} eq ‘valid’)
            {
            foreach $org (keys %{$mr2org{$mr}})
              {
                $possibleOrgs{$org}{numOfPeaksOnChart}++;
              }
            }
        }
      # calculate the percentage with which the peaks generated by an
organism from
      #the set of all possible organisms can be identified.
      foreach $org (keys %possibleOrgs)
        {
          $possibleOrgs{$org}{possibilityToBeInSample} =
$possibleOrgs{$org}{numOfPeaksOnChart} / (scalar keys
%{$org2mr{$org}});
        }
      print “\n”;
      foreach $org (sort {$possibleOrgs{$a}{possibilityToBeInsample}
      <=>
$possibleOrgs{$b}{possibilityToBeInSample} ∥ $a cmp $b} keys
%possibleOrgs)
        {
          if ($possibleOrgs{$org}{possibilityToBeInSample} > 0)
            {
            $prob = $possibleOrgs{$org}{possibilityToBeInSample}
            *100;
            $numOfPeaksOnChart = $possibleOrgs{$org}
            {numOfPeaksOnChart};
            $numOfPeaks = scalar keys %{$org2mr{$org}};
            write(STDOUT);
            }
          #print “$org\t”, $possibleOrgs{$org}*100, “\n” if
($possibleOrgs{$org} > 0.9);
        }
      print “\n--------------------------------------\n”;
      print “Peak width: $width\n”;
      print “Number of all peaks on MS chart: ”, scalar keys %mrChart,
      “\n”;
      print “These peaks are disqualified:\n”;
      $i = 0;
      foreach $mr (sort{$a <=> $b} keys %mrChart)
        {
          if ($mrChart{$mr} eq ‘invalid’)
            {
            print “$mr ”;
            $i++
            }
        }
      print “[$i]\nOrganisms in sample (“, scalar keys %orgsInSample,
”):\n\n”;
      foreach $orgInSample (sort {$a cmp $b} keys %orgsInSample)
        {
          print “$orgInSample\n”;
        }
      %orgsInSample = %mrChart = %possibleOrgs = ( );
      }
    }
format STDOUT =
@<<<<<<<<<< @###.##% @## /@##
$org, $prob, $numOfPeaksOnChart, $numOfPeaks
.
```

Because mass spectrometry differentiates oligonucleotides according to their molecular weights, instead of their compositions, this pool of oligonucleotides is in turn mapped into a collection of molecular weights. Each molecular weight in this collection may be attributed to a number of organisms whose 16S rRNAs digested by the RNase can generate one or several different oligonucleotides of the same molecular weight. The entire set of organisms identified by all the molecular weights and the number of times with which each of the organisms is identified are recorded. The probability that an organism is present in the sample is calculated as the ratio of the frequency with which it is identified to the number of oligonucleotides of different molecular weights in its RNase T1 catalogue of 16S rRNA. In the end, the program gives the list of all the organisms that are probably present in the sample and the corresponding probabilities.

The width of the peak in the MALDI-TOF mass spectrum establishes the resolution limitation of mass spectrometry. If two or more peaks are too close they will merge into a broad peak from which an accurate mass determination is not possible. This resolution problem is simulated by expunging molecular weights that are closer than a preset resolution threshold.

In an in silico experiment a simulated spectrum was produced under the assumption that a pool of 16S rRNA was isolated from a sample containing three organisms (Caulobacter intermedius str. CB63 ACM 2608; Metallosphaera sedula IFO 15509, and Oscillatoria agardhii str. CYA 18) was digested with RNase T1. The peak width threshold was assumed to be zero (This means that all peaks do not have width—they are atomic, which is only the ideal case.). A search of the database found that the top five organisms with highest probabilities to be present in the sample were Brevundimonas vesicularis LMG 2350, (96.25%), C. intermedius str. CB63ATCC 15262(96.25%), C. intermedius CB63 ACM 2608 (100%), M. sedula (100%) and O. agardh (100%). As we can see, all three organisms in the sample are correctly identified with 100% probability to be present in the sample by the program. The organisms found as high probability matches are closely related strains. The phylogenetic resolution of the method is dependent on the rRNA being used. If strains are indistinguishable by 16S rRNA sequence they will be indistinguishable by mass spectrometry of 16S rRNA T1 fragments too as is well understood [Fox et al., 1992 Fox, G E, Wisotzkey, J D, Jurtshuk, P Jr., “How Close is Close: 16S rRNA Sequence Identity may not be Sufficient to Guarantee Species Identity,” Intn. J. Syst. Bact. 1992:; 42: 166-170].

When this mass spectrometry approach is utilized in conjunction with rRNA it has the same properties as a comparison of the sequences themselves but with somewhat reduced resolution. Thus, just as there are signature sequences in the rRNA dataset [Zhang et al., Bioinformatics, 2002], the vast majority of the large fragments (greater than ten residues) produced by a RNAse T1 digestion also carry significant signature information. Thus, some peaks will be highly characteristic of particular bacterial groups. Thus, the spectra will in some instances contain peaks that are highly characteristic of particular phylogenetic groupings. Such peaks may be especially useful in characterizing complex mixtures of organisms.

The process of microbial identification by MALDI-TOF mass spectrometry using 16S rRNA endoribonuclease-generated catalogues can be simulated by a computer program and the effectiveness of this methodology as described above has been demonstrated by the results of such simulations. The utility of mass analysis of mixtures of characteristic oligonucleotides in microbial identification has been demonstrated by the disclosure described herein. Approximately one-sixth of the known major bacterial groupings can be identified based on the mass of a single unique rRNA fragments derived from endoribonuclease T1 digestion, and most organisms can be identified by a combination of fragments even in the absence of any knowledge of what might be in a sample. For example if medical specimen were being assayed, the presence of a mass peak characteristic of the pathogenic genera Chlamydia or the hot spring organism Sulfolobus would be unambiguous in this context.

As indicated by the in silico example presented here, identification of multiple species in mixtures is feasible. Practicable applicability of the method takes advantage of high performance mass spectrometric identification of the compositions of the characteristic oligonucleotides through accurate mass determination. Matrix assisted laser desorption ionization-time of flight (MALDI-TOF)MS offers sufficient resolution in size ranges which encompass most characteristic oligonucleotides observed in this study (3000-6000Da), and with sufficient precision under favorable conditions. Further advances in instrumentation will make the technique more powerful, less expensive, and more amenable to field applications. Quantization of the relative abundance of organisms in mixtures depends on the complexities of transfer of characteristic oligonucleotides to the gas phase, but transfer efficiencies for oligonucleotides of similar sizes are normally comparable, raising the possibility of at least semi-quantitative analysis of mixtures.

Mass spectrometry is not the only means of determining the composition of characteristic oligonucleotides which could be contemplated. In particular, analysis of stable isotope-labeled nucleotides in PCR fragments (e.g., by accelerator mass spectrometry or ion cyclotron resonance mass spectrometry, or even by capillary electrophoresis) is also possible.

The method will become more powerful as the size of the RNA databases increases. While the fraction of characteristic oligonucleotides, which is unique in the database will slowly decline as the entirety of the microbial world is covered, the use of multiple fragments for identification of organisms and understanding of the sample context will address this difficulty. Furthermore, because the sequence database was sufficiently large (n=1,921 starting sequences) it is likely that the number of informative compositions (masses) will remain similar on a percentage basis. In other words it shows that under appropriate conditions, certain molecules are informative “ICMs” and not random distributions of compositions or sequences.

The resolution of the technique is not exclusively dependent on the instrumentation. For example, amplification techniques might be used to increase the signal when sample is scarce or background contamination is likely to be a problem. This can be accomplished by amplifying a local region of the target RNA that carries one or more signature sequences. A particular advantage of amplification techniques is that the targeted amplification of informative subregion(s) of the target RNA eliminates competing fragments from the remainder of the sequence. Since the approach converts the target RNA to cDNA, restriction endonuclease digestion (typically with one or more enzymes recognizing sequences of only four bases) can subsequently be used to generate characteristic DNA oligonucleotides. This approach may be most promising when applied to mixed digests. An alternative would be to convert the cDNA back to RNA with the characteristic fragments subsequently released by chemical or enzymatic digestion. The conversion to RNA can be routinely accomplished by T7 runoff transcription or some other suitable technique. Finally, amplification techniques that produce an RNA product may also be used to generate large quantities of RNA segments containing signature sequences.

With the advent of artificial stable RNAs (aRNA) [Pitulle, C, Hedenstierna, KOF, Fox, G E “Artificial Stable RNAs: A Novel Approach for Monitoring Genetically Engineered Microorganisms,” Appl. Env. Micro. 1995; 61: 3661-3666 (1995).] it is possible to introduce “labeling” sequences into microbial rRNAs. These labeled aRNA molecules accumulate to high levels in the host without significantly perturbing its physiology. Labels can be selected to be unique in the background of interest, and a variety of different labels can be introduced into a single host for different applications. Labels could readily be designed to produce characteristic oligonucleotides of unique composition, and work in this direction is under way.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

1. A method for identifying or detecting organisms such as bacteria, eukaryotes, archaebacteria, or viruses comprising:
isolating a characteristic nucleic acid or protein component of an organism,
determining at least a portion of the monomer or molecular composition of a sequence derived from said characteristic nucleic acid or protein; and
identifying or detecting the micro-organism from which said characteristic nucleic acid or protein was derived by reference to a database of compositions of nucleic acids and proteins produced by organisms.

2. The method of claim 1 in which the characteristic molecule is DNA encoding ribosomal RNA or a fragment thereof.

3. The method of claim 1 in which the characteristic molecule is a protein or fragment thereof.

4. The method of claim 1 in which the characteristic molecule is a DNA encoding a protein or fragment thereof.

5. The method of claim 1 in which the composition is determined by mass spectrometry.

6. The method of claim 5 in which the method of mass spectrometry comprises matrix assisted laser desorption ionization (MALDI).

7. A system for identifying or detecting organisms such as bacteria, viruses, archaebacteria or eukaryotes comprising:
a chemical isolator or amplifier for identifying the characteristic nucleic acid or protein of an organism present in a specimen;
a controlled fragmentation reactor that generates sub-fragments of said characteristic acid or protein;
a mass spectrometer that measures the molecular weight of said sub-fragments and generates a set of representative data;
a computer that processes said data and compares said measured weights with known predicted sub-fragment masses to make an identification.

8. The system of claim 7 in which the characteristic molecule has been amplified by PCR, RT-PCR, LCR, NASBA, or Eberwine-type methods.

9. The system of claim 7 where the predicted sub-fragment masses are obtained from Genbank.

10. The system of claim 7 in which ribosomal RNA is isolated from a sample

11. The system of claim 7 in which the mass of the signature is determined within 0.01%.

12. The system of claim 7 wherein said mass spectrometry comprises matrix assisted laser desorption ionization (MALDI).

13. A method for identifying or detecting organisms such as bacteria, eukaryotes, archaebacteria, or viruses comprising:
determining known fragment sequences for a pre-determined set of nucleic acid or proteins;
isolating a characteristic nucleic acid or protein component of an organism present in a specimen,
determining at least a portion of the monomer composition of a sequence derived from said characteristic nucleic acid or protein; and
identifying or detecting the micro-organism from which said characteristic nucleic acid or protein was derived by reference to a database of compositions of nucleic acids and proteins produced by organisms.

14. The method of claim 13 in which the characteristic molecule is DNA encoding ribosomal RNA or a fragment thereof

15. The method of claim 13 in which the characteristic molecule is a protein or fragment thereof.

16. The method of claim 13 in which the characteristic molecule is a DNA encoding a protein or fragment thereof.

17. The method of claim 13 in which the composition is determined by mass spectrometry.

18. The method of claim 13 in which the method of mass spectrometry comprises matrix assisted laser desorption ionization (MALDI).
